(19) United States
(12) Patent Application Publication
(10) Pub. No.: US 2024/0353400 A1
(43) Pub. Date: Oct. 24, 2024

(54) BIOCOMPATIBLE SURFACE FOR QUANTUM SENSING AND METHODS THEREOF

(71) Applicant: The University of Chicago, Chicago, IL (US)

(72) Inventors: Peter C. Maurer, Chicago, IL (US); Mouzhe Xie, Chicago, IL (US); Xiaofei Yu, Chicago, IL (US)

(21) Appl. No.: 18/579,702

(22) PCT Filed: Jul. 15, 2022
(86) PCT No.: PCT/US2022/037386

§ 371 Date: Jan. 16, 2024

Related U.S. Application Data
(60) Provisional application No. 63/203,315, filed on Jul. 16, 2021.

(51) Int. Cl. G01N 33/543; B01L 3/00

(52) U.S. Cl. CPC G01N 33/54386; B01L 3/502707; B01L 2200/14; B01L 2200/16; B01L 2300/0663

(57) ABSTRACT

The present disclosure relates to a device having various layers that are supported on a substrate having one or more color centers. Such layers can include one or more capture agents configured to capture a target. Methods of making and using such devices are also described herein.

### STATEMENT OF GOVERNMENT INTEREST

This invention was made with Government support under Contract Nos. OMA-1936118 and OIA-2040520 awarded by the National Science Foundation. The Government has certain rights in the invention.

### BACKGROUND

Quantum spectroscopy can provide highly precise measurements of small ensembles of biomolecules or individual biomolecules. Such measurements can include nanoscale electron paramagnetic resonance (EPR) and nuclear magnetic resonance (NMR) analysis of proteins, nucleic acids, and the like.

### SUMMARY

The present disclosure relates to devices and methods that allow for quantum sensing of a target (e.g., a biological target). In particular examples, the device includes one or more color centers in proximity to a top surface, which in turn can be biocompatible. In other examples, the top surface is functionalized to selectively capture one or more targets. Methods of making and using such devices are also described herein.

In one aspect, the present disclosure provides a device including: a substrate having a top surface, wherein the substrate further includes one or more color centers in proximity to the top surface; and a functionalized layer configured to contact a sample. In further embodiments, the functionalized layer includes one or more capture agents configured to capture a target (e.g., any described herein).

In particular non-limiting examples, the substrate includes a diamond, and the one or more color centers include a nitrogen vacancy in the diamond. In one non-limiting embodiment, the one or more color centers are disposed at a depth of less than about 100 nm (e.g., or any range of depths herein) from the top surface.

In some embodiments, the device further includes an adhesion layer disposed on the top surface of the substrate, wherein the adhesion layer includes an oxide (e.g., a silanizable oxide, an aluminum oxide, a silicon oxide, a titanium oxide, a patterned oxide, or the like). The adhesion layer can be disposed on a portion of the top surface of the substrate or on a majority of the top surface of the substrate. In some non-limiting instances, the adhesion layer can be disposed on substantially the entirety of the top surface of the substrate. Optionally, the adhesion layer itself can be patterned, thereby providing a patterned adhesion layer having exposed portions (thereby providing exposed regions of the underlying substrate) and non-exposed portions (thereby providing covered regions overlying the substrate, in which the covered regions are composed of the material for the adhesion layer).

In other embodiments, the device further includes an interlayer disposed between the adhesion layer and the functionalized layer. The interlayer, in turn, can be disposed on a portion of the top surface of the adhesion layer, on a majority of the top surface of the adhesion layer, or on substantially the entirety of the top surface of the adhesion layer.

In some embodiments, the device can be characterized as having an active area and an inactive area. In one non-limiting instance, the active area includes one or more active sites, and the inactive area lacks active sites. In another non-limiting instance, the active area includes the functionalized layer and the adhesion layer, and the inactive area lacks the functionalized layer. In further instances, the inactive layer can also lack the adhesion layer.

In other embodiments, the device can further include: a source configured to irradiate the substrate and/or the one or more color centers; and a detector configured to detect one or more output signals emitted from the substrate upon being irradiated.

The present disclosure also provides a method of detecting a target. Accordingly, in one aspect, the method includes: providing a sample to an active area of a device (e.g., described herein); irradiating the device to excite the one or more color centers; and detecting one or more output signals emitted from the substrate upon being irradiated.

The present disclosure also provides a method of preparing a device (e.g., any device described herein). Accordingly, in one aspect, the method includes: depositing an adhesion layer on a top surface of a substrate, wherein the substrate comprises one or more color centers in proximity to the top surface; reacting a top surface of the adhesion layer with a silanizing agent to provide an interlayer, wherein a top surface of the interlayer comprises a reactive moiety; and attaching a functionalized layer by way of the reactive moiety within the interlayer, wherein the functionalized layer comprises one or more capture agents configured to capture a target.

In some embodiments, the adhesion layer can include an oxide (e.g., any described herein, including a silanizable oxide, a patterned oxide, or a combination thereof).

In some embodiments, said depositing includes atomic layer deposition, chemical vapor deposition, physical vapor deposition, or a plasma-enhanced form thereof.

In particular embodiments, said attaching includes providing a linking group (e.g., a poly(ethylene glycol) group, a perfluoroalkylene group, a perfluoroalkyleneoxy group, an alkylene group, a fluoroalkylene group, or a heteroalkylene group) that optionally includes one or more capture agents.

In other embodiments, said attaching includes providing a precursor (e.g., having a first linking group and a first reactive moiety configured to react with the reactive moiety of the interlayer). In particular embodiments, the first precursor includes a second reactive moiety configured to attach to a capture agent, or the first precursor includes a capture agent. In further embodiments, said attaching includes: providing one or more capture reagents to react with the second reactive moiety of the first precursor.

In some embodiments, said attaching includes providing a mixture of a first linking group (e.g., a poly(ethylene glycol) group) including the one or more capture agents and a second linking group (e.g., a poly(ethylene glycol) group) that lacks the one or more capture agents.

In some embodiments, said attaching includes: providing a mixture of a first precursor (e.g., having a first linking group and a first reactive moiety configured to react with the reactive moiety of the interlayer) and a second precursor (e.g., having a second linking group and a first reactive moiety configured to react with the reactive moiety of the interlayer). In particular embodiments, the first precursor includes a second reactive moiety configured to attach to a capture agent, or the first precursor includes a capture agent. In other embodiments, the second precursor includes a second moiety configured to not attach to a capture agent, or the second precursor lacks a capture agent. In further embodiments, said attaching includes: providing one or more capture reagents to react with the second reactive moiety of the first precursor.

In some embodiments, said attaching includes: providing a first linking group (e.g., a poly(ethylene glycol) group) having a further reactive moiety and providing one or more capture reagents to react with the further reactive moiety.

In other embodiments, said attaching includes: providing a mixture of a first linking group (e.g., a poly(ethylene glycol) group) having a further reactive moiety and a second linking group (e.g., a poly(ethylene glycol) group) that lacks the further reactive moiety, and providing one or more capture reagents to react with the further reactive moiety.

In some embodiments, said attaching includes: providing a mixture of a first precursor (e.g., having a first linking group, a first reactive moiety configured to react with the reactive moiety of the interlayer, and a second reactive moiety configured to attach to a capture agent) and a second precursor (e.g., having a second linking group, a first reactive moiety configured to react with the reactive moiety of the interlayer, and a second moiety configured to not attach to a capture agent). In further embodiments, said attaching includes: providing one or more capture reagents to react with the second reactive moiety of the first precursor.

### Definitions

As used herein, the term “about” means+/−10% of any recited value. As used herein, this term modifies any recited value, range of values, or endpoints of one or more ranges.

As used herein, the terms “top,” “bottom,” “upper,” “lower,” “above,” and “below” are used to provide a relative relationship between structures. The use of these terms does not indicate or require that a particular structure must be located at a particular location in the apparatus.

By “fluidic communication,” as used herein, refers to any duct, channel, tube, pipe, chamber, or pathway through which a substance, such as a liquid, gas, or solid may pass substantially unrestricted when the pathway is open. When the pathway is closed, the substance is substantially restricted from passing through. Typically, limited diffusion of a substance through the material of a plate, base, and/or a substrate, which may or may not occur depending on the compositions of the substance and materials, does not constitute fluidic communication.

By “alkoxy” is meant —OR, where R is an optionally substituted alkyl group, as described herein. Exemplary alkoxy groups include methoxy, ethoxy, butoxy, trihaloalkoxy, such as trifluoromethoxy, etc. The alkoxy group can be substituted or unsubstituted. For example, the alkoxy group can be substituted with one or more substitution groups, as described herein for alkyl. Exemplary unsubstituted alkoxy groups include C₁₋₃, C₁₋₆, C₁₋₁₂, C₁₋₁₆, C₁₋₁₈, C₁₋₂₀, or C₁₋₂₄ alkoxy groups.

By “alkyl” and the prefix “alk” is meant a branched or unbranched saturated hydrocarbon group of 1 to 24 carbon atoms, such as methyl (Me), ethyl (Et), n-propyl (n-Pr), isopropyl (i-Pr), cyclopropyl, n-butyl (n-Bu), isobutyl (i-Bu), s-butyl (s-Bu), t-butyl (t-Bu), cyclobutyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic (e.g., C₃₋₂₄ cycloalkyl) or acyclic. The alkyl group can be branched or unbranched. The alkyl group can also be substituted or unsubstituted. The alkyl group can be a primary, secondary, or tertiary alkyl group substituted with one or more substituents (e.g., one or more halo, alkoxy, amino, cyano, aryl, carboxyl, carboxyaldehyde, hydroxyl, nitro, amido, oxo, and the like). In some embodiments, the unsubstituted alkyl group is a C₁₋₃, C₁₋₆, C₁₋₁₂, C₁₋₁₆, C₁₋₁₈, C₁₋₂₀, or C₁₋₂₄ alkyl group.

By “alkylene” is meant a multivalent (e.g., bivalent) form of an alkyl group, as described herein. Exemplary alkylene groups include methylene, ethylene, propylene, butylene, etc. In some embodiments, the alkylene group is a C₁₋₃, C₁₋₆, C₁₋₁₂, C₁₋₁₆, C₁₋₁₈, C₁₋₂₀, C₁₋₂₄, C₂₋₃, C₂₋₆, C₂₋₁₂, C₂₋₁₆, C₂₋₁₈, C₂₋₂₀, or C₂₋₂₄ alkylene group. The alkylene group can be branched or unbranched. The alkylene group can also be substituted or unsubstituted. For example, the alkylene group can be substituted with one or more substitution groups, as described herein for alkyl.

By “amino” is meant —NR^{N1}R^{N2}, where each of R^{N1} and R^{N2} is, independently, H, optionally substituted alkyl, or optionally substituted aryl, or R^{N1} and R^{N2}, taken together with the nitrogen atom to which each are attached, form a heterocyclyl group.

By “aryl” is meant a group that contains any carbon-based aromatic group including, but not limited to, phenyl, benzyl, anthracenyl, anthryl, benzocyclobutenyl, benzocyclooctenyl, biphenylyl, chrysenyl, dihydroindenyl, fluoranthenyl, indacenyl, indenyl, naphthyl, phenanthryl, phenoxybenzyl, picenyl, pyrenyl, terphenyl, and the like. The term aryl also includes heteroaryl, which is defined as a group that contains an aromatic group that has at least one heteroatom incorporated within the ring of the aromatic group. Examples of heteroatoms include, but are not limited to, nitrogen, oxygen, sulfur, and phosphorus. The aryl group can be substituted or unsubstituted.

By “fluoroalkylene” is meant a bivalent form of an alkylene group, as defined herein, substituted with one, two, three, four, or more fluorine atoms.

By “halo” is meant F, Cl, Br, or I.

By “heteroalkylene” is meant a bivalent form of an alkylene group, as defined herein, containing one, two, three, or four non-carbon heteroatoms (e.g., independently selected from the group consisting of nitrogen, oxygen, phosphorous, sulfur, silicon, selenium, or halo). The heteroalkylene group can be substituted or unsubstituted.

By “perfluoroalkylene” is meant a bivalent form of an alkylene group, as defined herein, substituted completely with fluorine atoms.

### BRIEF DESCRIPTION OF THE DRAWINGS

FIG. 1A-1B is a set of schematics of (A) a non-limiting device 100 and (B) such a device in the presence of a target 160, 165.

FIG. 2 is a schematic illustration of a non-limiting method for preparing a device according to certain embodiments of the present disclosure. The method includes deposition of a layer of Al₂O₃ (or another material) on a pristine, oxygen-terminated diamond surface; silanization to provide a terminal reactive group (e.g., NH₂ here); and passivation (e.g., PEGylation) of the surface. The functional groups (circle and triangle) allow for cross-linking with target biomolecules. Optionally, the surface can be reset (e.g., by using of KOH and acid cleaning).

FIG. 3 is a schematic illustration of another non-limiting method for preparing a device according to certain embodiments of the present disclosure. The method includes deposition of a layer of TiO₂ (or another material) on a pristine, oxygen-terminated diamond surface; silanization to provide a terminal reactive group (e.g., NH₂ here); and passivation (e.g., PEGylation) of the surface. The functional groups (circle and triangle) allow for cross-linking with target biomolecules. Optionally, the surface can be reset (e.g., by boiling in concentrated sulfuric acid or NanoStrip).

FIG. 4 is a set of atomic force microscopy (AFM) images of surfaces according to certain embodiments of the present disclosure.

FIG. 5 includes (top) an AFM image of a lithographically fabricated Al₂O₃ pattern on a surface described herein by lift-off and (bottom) a plot showing surface height across a cross-section of the surface, showing a thickness of ˜2.1 nm. The Al₂O₃ layer was uniform, without the presence of pin holes. The elevated edges originated from lift-off combined with ALD.

FIG. 6 is a plot of water contact angle measurements of a surface according to certain embodiments of the present disclosure. Error bars represent standard deviation from four repeated measurements. Sample was water-rinsed and oven-dried between measurements. Inset: drop shape on a PEGylated diamond surface with the ellipse model used to extract the contact angle.

FIG. 7 is a set of graphs showing the dissolution rate of Al₂O₃ in 1M KOH (left) or 10 mM KOH (right), as measured by atomic force microscopy (AFM), from a surface according to certain embodiments of the present disclosure. The AFM measurements were done on the same features at four distinct locations (circles and dashed lines). The mean values are indicated by the solid lines.

FIG. 8 is a set of X-ray photoelectron scattering (XPS) spectra of surfaces according to certain embodiments of the present disclosure, XPS spectra of the Al2p signal are provided for a pristine diamond surface, a surface after Al₂O₃ deposition, a surface after silanization, and a surface after PEGylation (from left to right).

FIG. 9 is a set of graphs showing angle-resolved XPS (ARXPS) data used to estimate thicknesses of an Al₂O₃ layer (left) and a PEG layer (right) according to certain embodiments of the present disclosure.

FIG. 10 is a 3D plot of the results of a statistical analysis of 1000 simulated 3-dimensional random coil chains, as described herein. In the example shown, N=77 (equivalent to average m.w. 3400), a=0.35 nm, and 200 chains are displayed.

FIG. 11 is a set of histograms showing results of a statistical analysis of 1000 simulated 3-dimensional random coil chains, including (left) the distance of the free end from the surface, (center) the distance of the free end from the origin, namely, end-to-end distance, and (right) the maximum distance of any part of each chain from the surface. Vertical lines indicate Dₛ (90^{th} percentile of the distance of the free end from the surface), D_{E} (mean value of the end-to-end distance), and D_{Max} (90^{th} percentile of the maximum distance of any part of each chain from the surface).

FIG. 12 is a set of single-molecule fluorescence images of surfaces according to certain embodiments of the present disclosure. Images were obtained in Antiface medium. Scale bar is 5 μm.

FIG. 13 is an illustration of a non-limiting strain-promoted azide-alkyne cycloaddition (SPAAC) reaction described herein.

FIG. 14 is an illustration of a non-limiting imaging configuration described herein.

FIG. 15 is a set of fluorescence microscopy images of (left) a silanized surface (amine-terminated, before PEGylation) and (center, right) an mPEG-passivated surface after incubation with Alexa 488 dye-labeled streptavidin (SA-488). Both scale bars indicate 5 μm.

FIG. 16 is a set of fluorescence images of SA-488 molecules immobilized onto surfaces according to certain embodiments of the present disclosure. Images are provided for various biotinPEG percentages (0, 0.02, 0.1, 0.5, and 2%) and two different Al₂O₃ thickness (50 nm, imaged in buffer, and 2 nm, imaged in a refraction index=1.42 Invitrogen Antifade medium).

FIG. 17 is a set of fluorescence images of SA-488 molecules immobilized onto surfaces according to certain embodiments of the present disclosure. The diamond surfaces were all functionalized with 1% biotinPEG, yet incubated with Alexa-488 labeled streptavidin solution at various concentrations. As expected, the more concentrated solution leads to larger grafting density. Scale bar is 5 m.

FIG. 18 is a graph showing the results of quantitative analysis on the grafting density of SA-488 on surfaces according to certain embodiments of the present disclosure. Two independent experiments were conducted, suggesting good reproducibility. Error bars are the standard deviation of the grafting density calculated based on three images (each has a field-of-view of approximately 2800 μm²) for each data point.

FIG. 19 is a set of single-molecule fluorescence images of SA-488 immobilized onto (left) surfaces according to certain embodiments of the present disclosure (right) and comparative surfaces described herein. Al₂O₃ layers of 50 nm thickness were used here. Samples were imaged in sodium phosphate buffer. Scale bar 10 μm.

FIG. 20 is a set of single-molecule fluorescence images with epi-fluorescence illumination of SA-488 immobilized onto surfaces according to certain embodiments of the present disclosure having different Al₂O₃ layer thickness (2, 5, 20, or 50 nm). Samples were imaged in sodium phosphate buffer. Scale bar is 5 μm.

FIG. 21 is a set of zoomed-in images of the boxed regions in FIG. 20.

FIG. 22 is a graph of the mean of apparent signal-to-noise ratio (SNR) of certain single-molecule fluorescence images described herein. Error bars denote one standard deviation.

FIG. 23 is a graph comparing the optical power emitted away from a diamond surface described herein (P₁) to the total power radiated (P₀) into all directions, including surface guiding modes.

FIG. 24 is a non-limiting illustration of surfaces according to certain embodiments of the present disclosure.

FIG. 25 includes (left) an illustration of an immobilization system described herein via biotin-streptavidin interaction and (right) a set of single-molecule fluorescence images of Cy3-ssDNA immobilized on a surface according to certain embodiments of the present disclosure.

FIG. 26 includes (left) a representative area of single-molecule fluorescence images of Cy3-ssDNA immobilized on a surface according to certain embodiments of the present disclosure and (right) time traces of five selected fluorescence spots.

FIG. 27 includes (left) a graph of 90 fluorescence intensity traces collected from a sample described herein and (right) a histogram of the average intensities over the first second from fluorescence intensity traces collected from a sample described herein.

FIG. 28 includes (left) an illustration of an immobilization system described herein via SPAAC and (right) a set of single-molecule fluorescence images of Cy3-ssDNA immobilized on a surface according to certain embodiments of the present disclosure.

FIG. 29 includes (left) a representative area of a single-molecule fluorescence image (200×200 pixel area) of Cy3-ssDNA immobilized on a surface according to certain embodiments of the present disclosure and (right) time traces of three selected fluorescence spots.

FIG. 30 includes (left) a graph of 88 fluorescence intensity traces collected from a sample described herein and (right) a histogram of the average intensities over the first second from fluorescence intensity traces collected from a sample described herein.

FIG. 31 includes (top) a graph showing the number of SA-488 molecules per 100-μm² area (circles) of a surface according to certain embodiments of the present disclosure, detected by single-molecule fluorescence microscopy as a function of storage time in sodium phosphate buffer over a course of 1 week, and (bottom) a set of representative single-molecule microscopy images on a 50-nm-thick Al₂O₃ layer. Each data point is based on three 2,800-μm² field-of-view area; error bars indicated one SD. Fit is an exponential decay.

FIG. 32 is a set of graphs showing the overall thicknesses of a surface according to certain embodiments of the present disclosure in H₂O (left) and sodium phosphate buffer (right) tracked by AFM over 1 week at room temperature. Four unique sites (circles of the same shade) were monitored for each sample, and the mean values were fitted to a linear model (line) to estimate dissolution rates.

FIG. 33 is an optical image of a surface according to certain embodiments of the present disclosure.

FIG. 34 includes (top) an AFM scan of the region indicated by a box in FIG. 33, and (bottom) a plot of the 2D profile of the AFM scan averaged along the short edge.

FIG. 35 is a set of graphs showing the overall thicknesses of a surface according to certain embodiments of the present disclosure in sodium phosphate buffer (pH 7.4, [NaH₂PO₄+Na₂HPO₄]=50 mM, [NaCl]=100 mM) tracked by AFM over 1 week at (left) 37° C. and at (right) 23.5° C. Four unique sites (circles of the same shade) were monitored for each sample, and the mean values were fitted to a linear model (line). The uncertainty (+) reflects 95% confidence interval from fitting the averages.

FIG. 36 is a set of fluorescence images of “cross” shaped Al₂O₃ patterns (2 nm thickness) on a surface according to certain embodiments of the present disclosure, which was immersed in buffer (50 mM sodium phosphate buffer, pH 7.4, with 100 mM NaCl) at room temperature, over a course of 4 days. Scale bar is 10 μm.

FIG. 37 is a representative time trace of coherence measured by a (YY-8)_{N=8} sequence before (light gray) and after (dark gray) functionalization for an NV center (depth 4.8 nm) present in a substrate according to certain embodiments of the present disclosure. T₂ times were based on the fitted, stretched exponential decays (solid lines).

FIG. 38 is a non-limiting illustration of the YY8 pulse sequence described herein.

FIG. 39 is a set of plots showing exemplary depth measurements of NV centers present in a substrate according to certain embodiments of the present disclosure.

FIG. 40 is a plot of T₂ measured by a (YY-8)_{N=8} pulse sequence (total of 64 π-pulses) against NV depth before (light gray) and after (dark gray) functionalization for NV centers present in a substrate according to certain embodiments of the present disclosure.

FIG. 41 is a confocal scan of near-surface NV centers (implantation energy 3 keV) present in a substrate according to certain embodiments of the present disclosure. The eight NV centers studied for their coherence times are marked by circles.

FIG. 42 is a graph showing T₂ times as a function of the number of π-pulses for NV number 2 (depth 4.2 nm, triangles) and NV number 7 (depth 9.2 nm, open circles) of FIG. 39, before (light gray) and after (dark gray) functionalization.

FIG. 43 is a plot of T₂ measured by spin-echo pulse sequence against NV depth before (dark gray) and after (light gray) functionalization for NV centers present in a substrate according to certain embodiments of the present disclosure. Solid lines are fits based on Eqs. 1 and 2.

FIG. 44 is a broadband noise spectrum across the frequency range of 0.05 to 10 MHz for NV number 7 identified in FIG. 41. All measurements were carried out at 1,750-G magnetic field strength.

FIG. 45 is a set of broadband noise spectra across the frequency range of 0.05 to 10 MHz for NVs identified in FIG. 41.

FIG. 46 is a plot of T₁ against depth before (dark gray) and after (light gray) functionalization for the set of NV centers identified in FIG. 41.

FIG. 47 is a graph showing analytical results for the root-mean-square magnetic field noise (Bis) that is experienced by an NV center of depth (d) below the surface of a substrate according to certain embodiments of the present disclosure.

FIG. 48 is an XPS spectrum of the 2p electron signal of titanium (Ti2p) for a surface according to certain embodiments of the present disclosure before (dark gray) and after (light gray) an ALD coating described herein.

FIG. 49 is a plot of water contact angle measurements of a surface according to certain embodiments of the present disclosure.

FIG. 50 is a set of fluorescence images of surfaces described herein including Alexa488-dye-labeled streptavidin protein on (left) an mPEG-passivated surface or (right) a 0.3% biotinPEG-doped surface, which were on TiO₂-coated glass coverslips.

FIG. 51 is a set of atomic force microscopy (AFM) images of surfaces according to certain embodiments of the present disclosure including streptavidin protein on (left) an mPEG-passivated surface and (right) a 50% biotinPEG-doped surface, which were demonstrated on TiO₂-coated silicon wafer chips.

FIG. 52 is a set of plots of T₁ and T₂ coherence times measured in objective oil and water (left) before and (right) after functionalization of a 20 nm-thick Al₂O₃, as described herein.

### DETAILED DESCRIPTION

Despite recent developments in quantum engineering and substrate processing, biologically meaningful quantum sensing on a nanometer scale remains elusive. For example, challenges related to immobilization of target biomolecules within the sensing range of a qubit sensor (e.g., 10 nm to 30 nm, for a highly coherent nitrogen vacancy (NV) qubit sensor) have hampered life-science applications of nanometer-scale nuclear magnetic resonance (NMR).

Conventional approaches to immobilization have failed to yield a sensor-biomolecule interface suitable for quantum sensing. For example, though hydrogen-terminated diamond surfaces can be chemically modified to form biologically stable surfaces, near-surface NV sensors are generally charge-unstable under hydrogen termination. And though oxygen-terminated diamond surfaces can provide charge-stable NV centers with exceptional coherence times within 10 nm from the diamond surface, such surfaces are generally difficult to functionalize. Diamond nanocrystals, though functionalizable, typically lack the coherence times necessary for quantum sensing.

The present disclosure relates to a device having various layers that are supported on a substrate having one or more color centers. Such devices can provide precise control over biomolecule capture (e.g., position and density) within the sensing range of a qubit sensor having near-surface coherence times suitable for quantum-sensing techniques such as NMR and electron paramagnetic resonance (EPR).

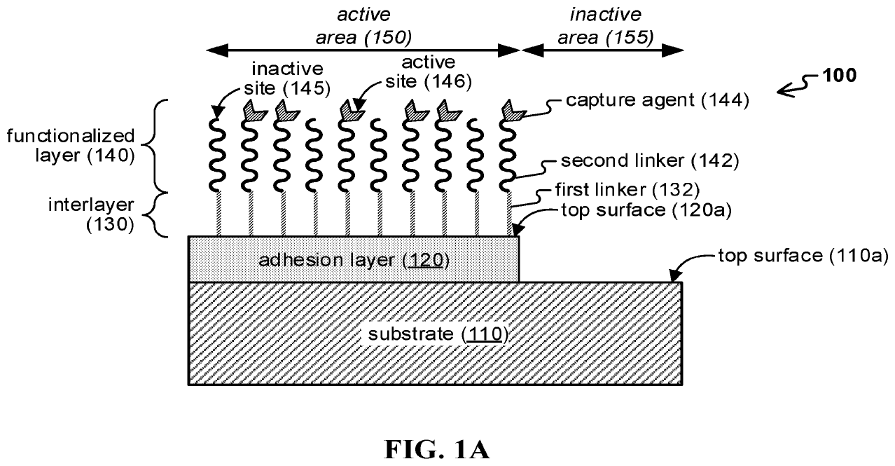

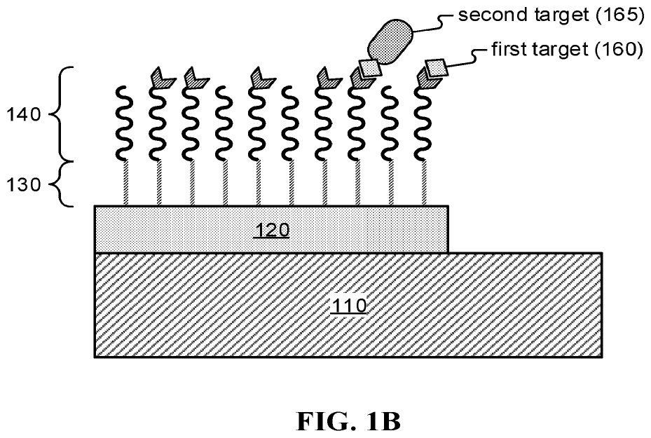

As seen in FIG. 1A, a non-limiting device 100 includes a substrate 110 having a top surface 110a; an adhesion layer 120 disposed on the top surface 110a; a functionalized layer 140 having inactive sites 145 lacking capture agents, as well as active sites 146 including one or more capture agents 144 configured to capture a target; and an interlayer 130 disposed between the adhesion layer 120 and the functionalized layer 140 or disposed on a top surface 120a of the adhesion layer 120.

In some embodiments, the substrate 110 further comprises one or more color centers in proximity to the top surface. Non-limiting color centers (e.g., defects) and substrate materials (e.g., crystalline materials) are described herein.

In particular embodiments, the adhesion layer 120 includes an oxide (e.g., an insulating oxide). The adhesion layer can provide a surface to which the interlayer and the functionalized layer can be attached. For example, the adhesion layer can include an oxide to which a silanizing compound (e.g., a silanizing compound described herein) can react (that is, a “silanizable oxide”). Such a silanizable oxide can include a functional group that can be reacted with a silanizing compound. Non-limiting examples of such functional groups include hydrogen (H), hydroxyl (OH), alkoxy (OR, in which R is an optionally substituted alkyl), aryloxy (e.g., OR<in which R is an optionally substituted aryl), halo, and the like. Such functional groups can be disposed, in some instances, on a surface portion of the adhesion layer for surface functionalization.

The adhesion layer can be disposed on all or a portion of the top surface of the substrate. In one instance, the adhesion layer can be disposed on substantially the entirety of the top surface of the substrate. In another instance, the adhesion layer can be disposed on a portion of the top surface of the surface. In one embodiment, the adhesion layer can be patterned (e.g., to provide a patterned layer), such as by use of lithography, etching, machining, milling, lift-off processes, and the like. When the adhesion layer includes an oxide that is patterned, then the adhesion layer can be considered a patterned oxide.

Furthermore, both the interlayer 130 and the functionalized layer 140 can be formed by using linkers 132, 142. Such linkers can include a silanizing compound, such as silazane (e.g., hexamethyldisilazane (HMDS)), haloalkylsilane (e.g., methyltrichlorosilane, trichlorocyclohexylsilane, dichlorodimethylsilane, dichloroethylsilane, bromotrimethylsilane, or chlorotrimethylsilane), haloarylsilane (e.g., fluorotriphenylsilane), trialkylsilylsilane (e.g., chlorotris(trimethylsilyl)silane), and silanol (e.g., 2-(trimethylsilyl)ethanol); a polyethylene glycol group (e.g., (CH₂CH₂O)ₙ, where n is from 1 to 50; or X—(CH₂CH₂O)ₙ—X, where n is from 1 to 50 and where each X is, independently, a reactive moiety); an alkylene group (e.g., an optionally substituted C₁₋₁₂ alkylene or alkynyl chain); a heteroalkylene group; a carbocyclic ring (e.g., an aromatic ring, such as a phenyl group); a polypeptide (e.g., a dipeptide, tripeptide, etc.); a flexible arm, e.g., 2, 3, 4, 5, 6, 7, 8, 9, 10, 11, 12, 13, 14, or 15 carbon atoms; BS3 ([bis(sulfosuccinimidyl) suberate], which is a homobifunctional N-hydroxysuccinimide ester that targets accessible primary amines, such as those present on proteins or antibodies); NHS/EDC (N-hydroxysuccinimide and N-ethyl-(dimethylaminopropyl)carbodiimide, in which NHS/EDC allows for the conjugation of primary amine groups with carboxyl groups); sulfo-EMCS ([N-e-maleimidocaproic acid]hydrazide, which are heterobifunctional reactive groups (maleimide and NHS-ester) that are reactive toward sulfhydryl and amino groups); hydrazide (most proteins contain exposed carbohydrates and hydrazide is a useful reagent for linking carboxyl groups to primary amines); and SATA (N-succinimidyl-S-acetylthioacetate, in which SATA is reactive towards amines and adds protected sulfhydryls groups). Other linkers and reactive moieties are described herein.

The functionalized layer 140 can include a mixture of two or more linkers 142. For example, the functionalized layer 140 can include a mixture of set of first linkers attached to a capture agent and a set of second linkers that lack a capture agent. In another example, the mixture can include a set of first linkers attached to a reactive moiety (which in turn can be used to attach a capture agent) and set of second linkers attached to an unreactive moiety (which does not attach to a capture agent under particular conditions). Such mixtures can be used to provide a corresponding mixture of inactive sites 145 (lacking a capture agent) and active sites 146 (having a capture agent). In this way, the density of active sites and inactive sites in the active area 150 can be controlled by the composition of these mixtures.

The device can possess an active area 150 that includes a functionalized layer (e.g., including inactive sites 145 and active sites 146) and an inactive area 155 that lacks the functionalized layer, e.g., and the interlayer and/or adhesion layer. In this way, the active area can be used for sensing and detection. The active area and inactive areas can be patterned, for example, in a configuration suitable for multiplexing or high-throughput applications.

In use, as seen in FIG. 1B, one or more capture agents in the functionalized layer 140 can be configured to capture a target 160, 165. As can be seen in FIG. 1B, the capture agent can bind to a sole target (as in the first target 160). Alternatively, the capture agent can bind to a tagged molecule (as in the second target 165) having a portion (diamond) that binds to the capture agent 144 and another portion (oval) that may be a further biomolecule, reporter, label, etc.

The device can have any useful feature or combination of features. In one instance, the thickness of each layer (either taken separately or together) can be configured to allow for optimal detection of output signals emitted from the substrate upon being irradiated. For example and without limitation, a thickness of the layer(s) disposed above the top surface of the substrate, when taken alone or together, can be less than about 50 nm (e.g., less than about 40, 30, 20, or 10 nm). In other instance, the thickness of the layer(s) disposed above the top surface of the substrate, when taken alone or together, is from about 1 nm to about 50 nm (e.g., from 1 to 40 nm, 1 to 30 nm, 1 to 20 nm, 1 to 10 nm, 1 to 5 nm, 2 to 50 nm, 2 to 40 nm, 2 to 30 nm, 2 to 20 nm, 2 to 10 nm, 2 to 5 nm, 3 to 50 nm, 3 to 40 nm, 3 to 30 nm, 3 to 20 nm, 3 to 10 nm, 3 to 5 nm, 5 to 50 nm, 5 to 40 nm, 5 to 30 nm, 5 to 20 nm, 5 to 10 nm, 10 to 50 nm, 10 to 40 nm, 10 to 30 nm, or 10 to 20 nm).

Such layer(s) can include only the functionalized layer. Alternatively, such layer(s) can include a combination of layers, such as a combination of the interlayer and the functionalized layer or a combination of the adhesion layer, the interlayer, and the functionalized layer.

The device can include one or more additional components to allow for detection of the target. For instance, the device can include a source (e.g., an optical source and/or a microwave source) configured to irradiate the substrate and/or the one or more color centers; and a detector (e.g., an optical detector) configured to detect one or more output signals emitted from the substrate upon being irradiated. Other components include filters, lenses, phase shifters, and the like.

### Substrate

The devices herein can employ any useful substrate. In one instance, the substrate include one or more color centers located in proximity to the top surface. Color centers generally include defects within transparent, crystalline insulators or large band-gap semiconductors, such as diamond, silicon carbide, germanosilicate glass, silica, or LiBaF₃. Such defects can include point defects; substitution defects in which an atom within the substrate is replaced with another atom; and vacancy defects in which an atom is missing within the crystalline lattice, as well as combinations thereof (e.g., nitrogen-vacancy (NV) centers in diamond having a nitrogen substitution in proximity to a vacancy, germanium-related detects in germanosilicate glass, silicon vacancies silicon carbide, and the like). Such color centers be probed via nanoscale nuclear magnetic resonance spectroscopy (NMR), optical detection of magnetic resonance (ODMR), or other techniques.

In some instances, the substrate is a diamond having one or more color centers at a depth of less than about 100 nm from the top surface of the substrate. Such color centers can be one or more NV centers. In other instances, at least one color center is at a depth of about 0.1 nm to about 100 nm from the top surface of the substrate (e.g., a depth of about 0.1 to 90 nm, 0.1 nm to 70 nm, 0.1 to 50 nm, 0.1 to 40 nm, 0.1 to 30 nm, 0.1 to 20 nm, 0.1 to 10 nm, 0.1 to 5 nm, 0.2 to 100 nm, 0.2 to 90 nm, 0.2 to 70 nm, 0.2 to 50 nm, 0.2 to 40 nm, 0.2 to 30 nm, 0.2 to 20 nm, 0.2 to 10 nm, 0.2 to 5 nm, 0.5 to 100 nm, 0.5 to 90 nm, 0.5 to 70 nm, 0.5 to 50 nm, 0.5 to 40 nm, 0.5 to 30 nm, 0.5 to 20 nm, 0.5 to 10 nm, 0.5 to 5 nm, 1 to 100 nm, 1 to 90 nm, 1 to 70 nm, 1 to 50 nm, 1 to 40 nm, 1 to 30 nm, 1 to 20 nm, 1 to 10 nm, 1 to 5 nm, 1.5 to 100 nm, 1.5 to 90 nm, 1.5 to 70 nm, 1.5 to 50 nm, 1.5 to 40 nm, 1.5 to 30 nm, 1.5 to 20 nm, 1.5 to 10 nm, 1.5 to 5 nm, 2 to 100 nm, 2 to 90 nm, 2 to 70 nm, 2 to 50 nm, 2 to 40 nm, 2 to 30 nm, 2 to 20 nm, 2 to 10 nm, 2 to 5 nm, 5 to 100 nm, 5 to 90 nm, 5 to 70 nm, 5 to 50 nm, 5 to 40 nm, 5 to 30 nm, 5 to 20 nm, or 5 to 10 nm, from the top surface of the substrate). In particular embodiments, the substrate is diamond having an oxygen-terminated surface and stable NV centers in proximity to this surface.

The substrate may include any useful structure, such as a planar structure, pillars, particles, and the like, which can be provided in any useful pattern.

Any portion of the top surface of the substrate may be covered by any layer described herein (e.g., an adhesion layer, a functionalized layer, and/or an interlayer). In some embodiments, only a portion of the top surface of the substrate is covered by a layer (e.g., any layer described herein). In other embodiments, a substantial portion or all of the top surface of the substrate is covered by a layer (e.g., any layer described herein).

### Adhesion Layer

The device can include an adhesion layer, which is disposed on a top surface of a substrate. In some embodiments, the adhesion layer includes an oxide, such as aluminum oxide, silicon oxide, or titanium oxide. The adhesion layer can be deposited in any useful manner, including chemical vapor deposition (CVD), atomic layer deposition (ALD, e.g., thermal ALD and plasma-enhanced ALD), physical vapor deposition (PVD), or molecular layer deposition (MLD), plasma-enhanced forms thereof, sputter deposition, e-beam deposition including e-beam co-evaporation, etc., or a combination thereof, such as ALD with a CVD component, such as a discontinuous, ALD-like process in which metal- or metalloid-containing precursors and oxygen-containing reactants are separated in either time or space.

To provide an oxide layer, deposition can include the use of a metal- or metalloid-containing precursor with an oxygen-containing reactant. For example, the metal- or metalloid-containing precursor and oxygen-containing reactant can be introduced at separate times, representing an ALD cycle. The precursor can react on the surface, forming up to a monolayer of material at a time for each cycle. An oxygen-containing reactant may be pulsed between the precursor pulses resulting in ALD or ALD-like growth of the oxide layer. In other cases, both the precursor and the oxygen-containing reactant may be flowed at the same time.

The adhesion layer may be patterned. In one instance, such patterning can include etching of the adhesion layer; immobilizing functional moieties (e.g., reactive or unreactive moieties) on a top surface of the adhesion layer; and the like.

### Interlayer

The device can include an interlayer disposed between the functionalized layer and the adhesion layer. Without wishing to be limited by mechanism, the interlayer can be used to control the homogeneity of the functionalized layer within the device. For instance and without limitation, as described herein, a device lacking the interlayer exhibited heterogenous distribution of the capture agents within the functionalized layer. Thus, in some instances, the composition and structure of the interlayer can be used to optimize the spatial homogeneity, and/or spatial density of the functionalized layer (or the capture agents within the functionalized layer).

To provide the interlayer, deposition can include the use of a reagent to react with a top surface of the adhesion layer. Such deposition can include conditions or reagents that provide a reactive surface upon which further layers can be attached to the interlayer. The reactive surface can include one or more reactive moieties, which are in turn provided by the reagent.

In one embodiment, the reagent is a silanizing agent having at least one reactive moiety to react with the adhesion layer and another reactive moiety to react with the functionalized layer. For example, the silanizing agent can be (R¹)₃—Si-Ak-R² or (R¹)₃—Si-Ak-NR^{N1}-Ak-R², in which each of R¹ and R² is, independently, a reactive moiety; each Ak is, independently, optionally substituted alkylene; and R^{N1} is H or optionally substituted alkyl. Non-limiting reactive moieties include H, halo, alkoxy, amino, optionally substituted alkyl, and the like. In particular embodiments, R¹ is not amino, and R² is or includes amino.

In particular embodiments, the interlayer includes an alkylene group (e.g., -Ak-, such as a C₁₋₁₂ alkylene) or a heteroalkylene group (e.g., -Ak-NR^{N1}—, -Ak-O—, [symbol] Si-Ak-NR^{N1}-Ak-NR^{N1}—, [symbol] Si-Ak-, [symbol] Si-Ak-NR^{N1}—, or [symbol] Si-Ak-O—, in which Ak is C₁₋₁₂ alkylene, R^{N1} is H or C₁₋₆ alkyl).

### Functionalized Layer

The device can include a functionalized layer, which is configured to contact with the sample. Thus, in some embodiments, the functionalized layer is biocompatible or is configured to provide a biocompatible surface to the sample. In other embodiments, the functionalized layer includes a monolayer.

In particular embodiments, the functionalized layer includes a linking group, such as a poly(ethylene glycol) group (e.g., —(CH₂CH₂O)ₙ—), a perfluoroalkylene group (e.g., —C_{f}F_{2f}—, in which f is an integer from about 1 to 12 and 2f is an integer that is 2 times f), a perfluoroalkyleneoxy group (e.g., —OC_{f}F_{2f}— or —C_{f}F_{2f}O—, in which f is an integer from about 1 to 12 and 2f is an integer that is 2 times f), an alkylene group, a fluoroalkylene group, or a heteroalkylene group. For example, the functionalized layer can include —(CH₂CH₂O)ₙ—, in which n is an integer of 5 to 200 (e.g., 5 to 15, or 25 to 150). Such linking groups can be provided as a precursor (e.g., having one or more reactive moieties to attach the linking group to a layer or a substrate).

To provide the functionalized layer, any useful precursor can be employed. Non-limiting precursors can include one or more monomer groups or other linking groups, such as those including an ethylene glycol group —OCH₂CH₂—, including a poly(ethylene glycol) (PEG) group —(OCH₂CH₂)ₙ—, or a derivatized PEG group (e.g., methyl ether PEG (mPEG), a propylene glycol group, etc.), including dendrimers thereof, copolymers thereof (e.g., having at least two monomers that are different), branched forms thereof, start forms thereof, comb forms thereof, etc., in which n is any useful number in any of these (e.g., any useful n to provide any useful number average molar mass Mₙ). The precursor can be a poly(ethylene glycol) group (e.g., a multivalent poly(ethylene glycol) precursor having one or more reactive moieties, such as an amino group, an ester group, an acrylate group, a hydroxyl group, a carboxylic acid group, a halo group, etc.). In one instance of a precursor for providing a functionalized layer, a first reactive moiety can react with the interlayer, and a second reactive moiety can participate in a reaction to immobilize the capture agent.

Any linking group herein can include one or more reactive moieties (e.g., as described herein, such as an amino group, an ester group, an acrylate group, a hydroxyl group, a carboxylic acid group, a halo group, etc.). Such reactive moieties can be configured to react with a reactive moiety of the interlayer, the adhesion layer, the top surface of the substrate, and/or the capture agent. In one instance, a precursor for the functionalized layer can include a first reactive moiety configured to react with the interlayer, a second reactive moiety configured to participate in a reaction to immobilize the capture agent, and a linking group disposed between the first and second reactive moieties.

In certain other instances, a precursor for the functionalized layer can include a first reactive moiety configured to react with the interlayer, a second non-reactive moiety, and a linking group disposed between the first and second reactive moieties. Such a non-reactive moiety can be used to control the density of desired capture agents to be provided in the functionalized layer. Non-limiting examples of non-reactive moieties include, e.g., hydrogen (H), an alkyl group, a haloalkyl group, an aryl group, and the like. As described herein, linking groups, reactive moieties, and non-reactive moieties can be combined to provide any useful precursor to provide the functional layers described herein.

As described herein, the precursor or the linking group can include at least one reactive moiety, such as to attach the precursor or the linking group

The functionalized layer can include one or more capture agents. One or more capture agents can be selected from the group of a nucleic acid (e.g., a nucleotide, a single stranded DNA, a single stranded RNA, and an oligonucleotide, including modified forms of any of these, as well as hairpin forms or double-stranded forms of these; also including a polythymine), a peptide (e.g., a polypeptide, including modified forms thereof, such as glycosylated polypeptides or multimeric polypeptides), a protein (e.g., avidin, streptavidin, neutravidin, an enzyme, a receptor, and the like), a cofactor (e.g., biotin or a metal ion such as Ni²⁺), a receptor, an enzyme, an antibody (e.g., including monoclonal or polyclonal forms thereof, an affibody, or fragments or recombinant forms of any of these), an affibody, a lectin, and a click chemistry moiety (e.g., an azido group, an alkynyl group, a dienophile group, or a diene group).

A peptide can include a polyhistidine (e.g., including 6-9 histidine residues) or a polyglycine (e.g., including 4-6 glycine residues). Other peptides may be employed, such as those that can be used as an affinity tag. Non-limiting affinity tags include a polyhistidine tag, a polyarginine tag (e.g., including 4-6 arginine residues), glutathione-S-transferase (GST), a FLAG tag (e.g., DYKDDDDK, SEQ ID NO:2), a streptavidin-binding protein, a streptavidin binding tag, a modified streptavidin-binding tag (e.g., WSHPQFEK, SEQ ID NO:3), a calmodulin binding peptide (CBP) tag, a chitin-binding domain (CBD) tag, maltose-binding protein (MBP) tag, as well as combinations thereof or modified forms thereof.

A click chemistry moiety can include those from a click-chemistry reaction pair selected from the group consisting of a Huisgen 1,3-dipolar cycloaddition reaction between an alkynyl group and an azido group to form a triazole-containing linker; a Staudinger reaction between an azido group and a phosphine or phosphite to form a iminophosphorane-containing linker; a Diels-Alder reaction between a diene having a 4π electron system (e.g., an optionally substituted 1,3-unsaturated compound, such as optionally substituted 1,3-butadiene, 1-methoxy-3-trimethylsilyloxy-1,3-butadiene, cyclopentadiene, cyclohexadiene, or furan) and a dienophile or heterodienophile having a 2π electron system (e.g., an optionally substituted alkenyl group or an optionally substituted alkynyl group); a ring opening reaction with a nucleophile and a strained heterocyclyl electrophile; a splint ligation reaction with a phosphorothioate group and an iodo group; a reductive amination reaction with an aldehyde group and an amino group; and a Michael addition reaction between a thiol group and a maleimide group, as well as variants of any of these.

In certain embodiments, the click chemistry moieties are conducted in a copper-free condition. In one example, a click chemistry moiety can include those from a click-chemistry reaction pair selected from the group consisting of a Huisgen 1,3-dipolar cycloaddition reaction between an cyclic alkynyl (or cycloalkynyl) group (e.g., a cyclooctynyl group, including optionally substituted forms thereof, such as halo substituted forms) and an azido group to form a triazole-containing linker. In certain embodiments, the cyclic alkynyl group can further include one or more heteroatoms (e.g., nitrogen, oxygen, sulfur, and the like).

The one or more capture agents can include moieties from one or more bioconjugate pairs, for example, one or more moieties configured to form a covalent link between a capture agent and a biomolecule. Bioconjugate pairs can include, for example, biotin and a biotin-binding biomolecule (e.g., avidin, streptavidin), maleimide and a thiol-containing biomolecule (e.g., a cysteine-containing biomolecule), a metal ion (e.g., Ni²⁺) and a histidine-containing biomolecule (e.g., a polyhistidine-tagged biomolecule), a polyglycine and a biomolecule containing a sortase signal (e.g., LPXTG, SEQ ID NO:4), and click chemistry pairs such as an azido group and an alkynyl-containing biomolecule (e.g., a DBCO-tagged biomolecule).

The capture agents can be distributed spatially homogeneously as a monolayer throughout the functionalized layer. In some embodiments, the average number of capture agents present per μm² of the functionalized layer is less than 10, or less than 7.5, or less than 5. For example, the functionalized layer can include an average of about 0.01 to about 10, about 0.01 to about 7.5, about 0.01 to about 5, about 0.1 to about 10, about 0.1 to about 7.5, about 0.1 to about 5, about 0.1 to about 2.5, or about 0.1 to about 1 capture agents per m². In other examples, the functionalized layer can include up to about 1,000, up to about 10,000, or up to about 100,000 capture agents per μm². In particular examples, the functionalized layer can include up to about 1 capture agent per nm².

### Targets

Methods herein can be used for detecting one or more targets. In some non-limiting embodiments, detection of the target is conducted in a label-free manner. For example, the target of interest can be free of labels, and selectivity for the target can be provided by the capture agent(s).

Non-limiting targets include a biomolecule (including a tagged biomolecule), a nucleic acid (e.g., e.g., oligonucleotides, polynucleotides, nucleotides, nucleosides, molecules of DNA, or molecules of RNA, including a chromosome, a plasmid, a viral genome, a primer, or a gene), a peptide, a protein, a receptor, a ligand, a toxin, a cell, a tissue, a bacterium, a virus, a pathogen, a microorganism, an allergen, as well as components thereof (e.g., a modification, a polymorphism, a structural configuration such as folding or misfolding configuration of a nucleic acid, a peptide, or a protein). In other embodiments, the target is a chemical, a small molecule, a pharmaceutical, and the like.

The target can be present in any useful sample. Non-limiting samples can include a microorganism, a virus, a bacterium, a fungus, a parasite, a helminth, a protozoon, a cell, tissue, a fluid, a swab, a biological sample (e.g., blood, serum, plasma, saliva, etc.), a plant, an environmental sample (e.g., air, soil, and/or water), etc.

### Other Components

The devices and methods herein can be used with any other useful component. Non-limiting components include a source (e.g., configured to provide radiation to the substrate, including excitation light, microwave radiation, and the like), a detector (e.g., configured to detect an optical emission from the substrate, an emitted radiation, and the like, as well as frequency and/or wavelength measurements), a fluidic device (e.g., configured to provide a sample or a target to a capture agent, such as a well, a microfluidic device, and the like), a sample holder, a manifold, and the like.

Non-limiting sources include a pulsed source (e.g., a pulsed optical source or a pulsed microwave source), a microwave/radiofrequency electromagnetic field source, an optical source (e.g., a laser or a light emitting diode), a microwave source (e.g., a tuned microwave source), and the like. Non-limiting detectors include a photodetector, an electronic detector, or an optoelectronic detector.

The fluidic device can include any fluidic structure configured to provide fluidic communication to a surface of the substrate. Such fluidic structures can include a channel, a well, a chamber, an access port, a reservoir, and the like.

### Methods

Methods herein include those for using or making a device, such as a device described herein. In one instance, the method includes detecting a target. Non-limiting targets includes a biomolecule or a tagged biomolecule. In particular embodiments, the biomolecule includes a nucleic acid, a peptide, a protein, a receptor, a ligand, or a cell. For detection, the device can include a capture agent that binds to the biomolecule, the tagged biomolecule, or a portion thereof. One non-limiting method includes providing a sample to an active area of a device (e.g., any described herein); irradiating the device to excite the one or more color centers (e.g., by use of a source); and detecting one or more output signals emitted from the substrate upon being irradiated (e.g., by use of a detector). In some embodiments, the sample provided to the active area comprises a biomolecule and/or a physiological buffer.

Methods of making the device are also described herein. Such a method can include, without limitation, depositing an adhesion layer on a top surface of a substrate (e.g., as described herein), wherein the substrate comprises one or more color centers in proximity to the top surface; reacting a top surface of the adhesion layer with a silanizing agent to provide an interlayer, wherein a top surface of the interlayer comprises a reactive moiety; and attaching a functionalized layer by way of the reactive moiety within the interlayer, wherein the functionalized layer comprises one or more capture agents configured to capture a target. In some embodiments, attaching the functionalized layer includes providing a poly(ethylene glycol) group optionally comprising the one or more capture agents. In other embodiments, attaching the functionalized layer includes providing a poly(ethylene glycol) group having a further reactive moiety, and then providing one or more capture reactants to react with the further reactive moiety.

Deposition of a material, as well as patterning or treating a layer of the material, can include any useful process. Exemplary processes include epitaxial growth; polishing, such as chemical-mechanical polishing (CMP); chemical vapor diffusion (CVD), such as metal-organic CVD (MOCVD), metal-organic vapor phase epitaxy (MOVPE), plasma-enhanced chemical vapor deposition (PECVD), and molecular beam epitaxy (MBE); milling (e.g., ion milling or focused ion beam milling); rapid prototyping; microfabrication (e.g., by casting, injection molding, compression molding, embossing, ablation, thin-film deposition, and/or Computer Numerically Controlled (CNC) micromachining); photolithography; atomic layer deposition (ALD); and etching techniques (e.g., wet chemical etching, reactive ion etching (RIE), deep RIE, sputter etching, inductively coupled plasma deep silicon etching, buffered oxide etching (BOE), laser ablation, or air abrasion techniques). In one instance, the adhesion layer can be constructed of any oxide material deposited via physical or electrochemical deposition, which can be optionally patterned or implanted.

A surface of a material can be further reacted or functionalized in any useful manner. In one instance, the surface can be reacted with an agent to provide a reactive moiety. Non-limiting agents can include silanizing agents, PEGylating agents, and the like. Such agents can provide a linker (e.g., to which additional moieties or capture agents can optionally be added), a reactive moiety, a capture agent, and the like.

Alternatively, the surface can be reacted with an agent to provide a biocompatible or cytocompatible moiety. Non-limiting examples of such agents include a biocompatible polymer, a biopolymer such as chitosan, a cationic polymer, an antifouling polymer, and the like. For instance and without limitation, biocompatible polymers can include poly(ethylene glycol); poly(lactic acid) (PLA) including poly(DL-lactic acid) (DL-PLA), poly(L-lactic acid) (L-PLA), and poly(D-lactic acid) (D-PLA); poly(glycolic acid) (PGA); poly(lactic-co-glycolic acid) (PLGA) including poly(DL-lactic-co-glycolic acid) (DL-PLGA); a poly(ester), such as polyhydroxybutyrate, polyhydroxyvalerate, or copolymers thereof; poly(vinyl alcohol); poly(dioxanone); poly(caprolactone); poly(orthoester); poly(anhydride); poly(phosphazine); poly(propylene carbonate); poly(propylene succinate); poly(urethane); as well as copolymers thereof),

In other embodiments, the agent can be poly(imide), benzocyclobutene, glass, a fluoropolymer (e.g., a fluoroacrylate or polytetrafluoroethylene), a photoresist, and the like.

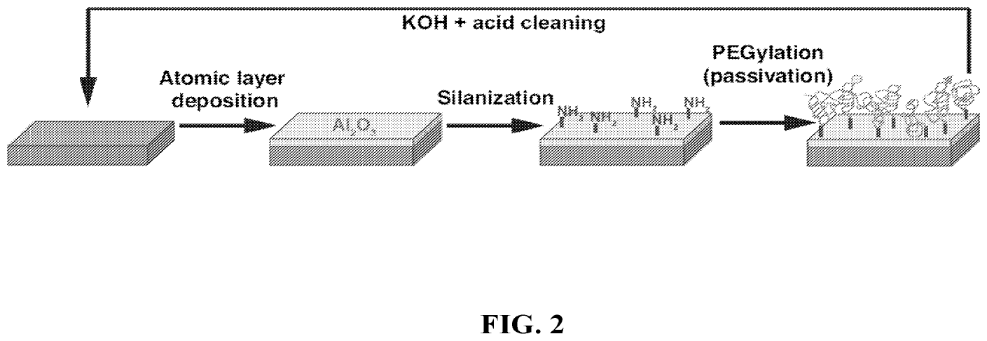

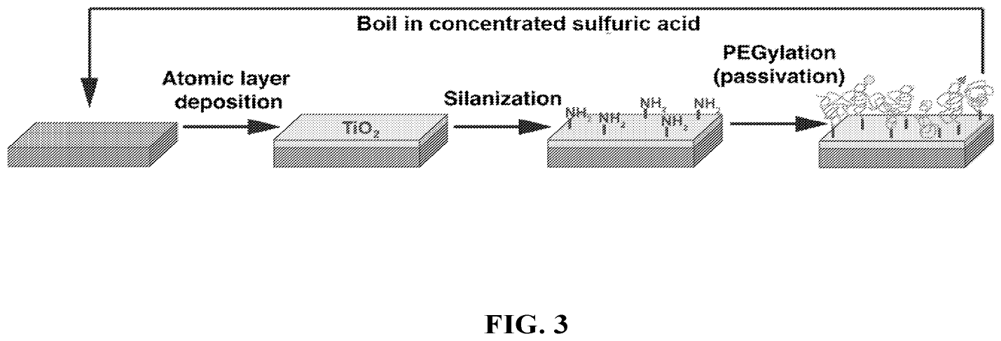

For example, in certain embodiments, the method includes atomic layer deposition of Al₂O₃ onto a pristine, oxygen-terminated diamond surface, followed by silanization (to provide a first linker attached to the diamond surface and to present a terminal reactive group), and then PEGylation (e.g., with a poly(ethylene glycol) group comprising the one or more capture agents, or with a poly(ethylene glycol) group having a further reactive moiety, in which the PEG group can react with the terminal reactive group provided by way of silanization), as illustrated in FIG. 2. In another example, in certain embodiments, the method includes atomic layer deposition of TiO₂ onto a pristine, oxygen-terminated diamond surface, followed by silanization and PEGylation (e.g., with a poly(ethylene glycol) group comprising the one or more capture agents, or with a poly(ethylene glycol) group having a further reactive moiety), as illustrated in FIG. 3.

### EXAMPLES

### Example 1: Biocompatible Surface Functionalization Architecture for a Diamond Quantum Sensor

Quantum metrology enables some of the most precise measurements. In the life sciences, diamond-based quantum sensing has led to a new class of biophysical sensors and diagnostic devices that are being investigated as a platform for cancer screening and ultrasensitive immunoassays. However, a broader application in the life sciences based on nanoscale NMR spectroscopy has been hampered by the need to interface highly sensitive quantum bit (qubit) sensors with their biological targets. The Examples herein demonstrate an approach that combined quantum engineering with single-molecule biophysics to immobilize individual proteins and DNA molecules on the surface of a bulk diamond crystal that hosts coherent nitrogen vacancy qubit sensors. The thin (sub-5 nm) functionalization architecture described below provided precise control over the biomolecule adsorption density and resulted in near-surface qubit coherence approaching 100 μs. The developed architecture remained chemically stable under physiological conditions for over 5 days, making the technique compatible with most biophysical and biomedical applications.

Recent developments in quantum engineering and diamond processing have brought us considerably closer to performing nanoscale NMR and electron paramagnetic resonance (EPR) spectroscopy of small ensembles and even individual biomolecules. Notably, these advances have allowed for the detection of the nuclear spin noise from a single ubiquitin protein and the probing of the EPR spectrum of an individual paramagnetic spin label conjugated to a protein or DNA molecule. More recently, lock-in detection and signal reconstruction techniques have allowed for one- and multidimensional NMR spectroscopy with 0.5-Hz spectral resolution. More advanced control sequences at cryogenic temperatures have further allowed mapping the precise location of up to 27 ¹³C nuclear spins inside of diamond. Yet biologically meaningful spectroscopy on intact biomolecules remains elusive. One of the main outstanding challenges, which is required to perform nanoscale magnetic resonance spectroscopy of biomolecules, is the need to immobilize the target molecules within the 10- to 30-nm sensing range of a highly coherent nitrogen vacancy (NV) qubit sensor. Immobilization can be important because an untethered molecule could otherwise diffuse out of the detection volume within a few tens of microseconds.

Various avenues to the functionalization of high-quality, single-crystalline diamond chips have been pursued over the last decade. Yet challenges remain for interfacing a coherent quantum sensor with target biomolecules. For example, hydrogen-terminated diamond surfaces can be chemically modified and form biologically stable surfaces; but near-surface NV centers are generally charge-unstable under hydrogen termination, posing open challenges for NV sensing. On the other hand, oxygen-terminated diamond surfaces have been used to create charge stable NV⁻ centers with exceptional coherence times within 10 nm from the diamond surface. However, perfectly arranged, ether-terminated diamond surfaces generally lack chemically functionalizable surface groups (such as carboxyl or hydroxyl groups), making it difficult to control immobilization density and surface passivation. Other platforms such as diamond nanocrystals can generally be functionalized because of their heterogeneous surface chemistry, but they do not possess the coherence times needed for nanoscale magnetic resonance spectroscopy.

In some non-limiting embodiments, the approaches herein can overcome these limitations by utilizing a thin (e.g., 2-nm-thick) oxide (e.g., TiO₂ or Al₂O₃) layer deposited onto an oxygen-terminated diamond surface in any useful manner (e.g., by atomic layer deposition (ALD)). This “adhesion” layer (e.g., TiO₂ or Al₂O₃) can be further reacted, e.g., by way of silanization to create a surface having a reactive moiety (e.g., an amine (—NH₂)-terminated surface). In turn, this surface can be grafted with a functionalized layer, such as, e.g., a monolayer of heterobifunctional polyethylene glycol (PEG) via an N-hydroxysuccinimide (NHS) reaction, a process also referred as PEGylation. Without wishing to be limited by mechanism, the PEG layer serves two purposes. First, it can passivate the diamond surface to prevent nonspecific adsorption of biomolecules. Second, by adjusting the density of PEG molecules with functional groups (e.g., biotin or azide), the immobilization density of proteins or DNA target molecules on the diamond surface can be controlled if desired. Furthermore, the small persistence length of the PEG linker (˜0.35 nm) allows the immobilized biomolecules to undergo rotational diffusion. This tumbling motion is the basis for motional averaging of the NMR spectra and can help to prevent immobilization of molecules in biologically inactive orientations.

Diamond-based quantum sensing allows for nanoscale measurements of biological systems with unprecedented sensitivity. Potential applications of this emerging technology range from the investigation of fundamental biological processes to the development of next-generation medical diagnostics devices. One challenge faced by bioquantum sensing is the need to interface quantum sensors with biological target systems. Specifically, such an interface needs to maintain the highly fragile quantum states of our sensor and at the same time be able to fish intact biomolecules out of solution and immobilize them on our quantum sensor surface. The examples herein addressed these challenges using tools from quantum engineering, single-molecule biophysics, and material processing.

The Examples herein demonstrate that a chemically stable, universal surface functionalization architecture could be combined with biochemical conjugation techniques including biotin-SA conjugation and SPAAC click chemistry. The functionalization approach can be readily extended to other conjugation techniques, such as a maleimide reaction, a Ni²⁺/His-tag interaction, a sortase-mediated enzymatic conjugation, and the like. Combined with single-molecule fluorescence imaging techniques, this architecture demonstrated a precise control over the conjugation density of individual target proteins and DNA molecules. The observed NV coherence times of up to 100 μs were long enough to perform highly sensitive, state-of-the-art quantum sensing experiments on biological targets. Based on the demonstrated sensor-target distances and qubit coherence, it was predicted that the NMR signal of an individual ¹³C nuclear spin could be detected with short integration times (e.g., as short as 100 s). Further strategies are described herein to improve integration time.

The Examples herein provide a process in which silanization is used on the surface of the substrate. Alternatively, silanization can be extended to directly conjugate —OH-terminated diamond surfaces, thereby eliminating the need for an adhesion layer. Such a technique can include the use of high-quality, —OH-terminated surface on (100) bulk diamond, in which some computational models have suggested that near-surface NV centers can remain charge-stable in the presence of —OH termination.

NV sensing can be used to probe the NMR signature of a self-assembled monolayer of organic molecules on an Al₂O₃-coated diamond sensors. Our molecular pulldown approach can be combined with NV based NMR and EPR spectroscopy techniques.

The devices herein can be employed with other structures (e.g., sample holders, flow cells, fluidic structures, and the like). For example, a microfluidic platform can readily be combined with the diamond passivation and functionalization methods herein. Fluidic components can be used to deliver samples, as well as to deliver reagents (e.g., capture agents, linkers, and the like) to provide active areas with desired patterns.

Arrays of the devices herein can be also be employed. For example, arrays can be configured for multiplexing or high-throughput applications. In other examples, heterofunctional arrays can be configured for detection of two or more targets (e.g., biological targets) in a sample. Arrays can include patterned regions having a plurality of active areas that are isolated by inactive areas, in which an individual active area can optionally be individually addressed.

Such configurations could enable label-free, high-throughput biosensing with various applications (e.g., quality management in the pharmaceutical industry, screening for targets in drug discovery), single-cell screening for metabolomics, proteomics, detection of cancer markers, and the like).

Furthermore, positioning individual biomolecules within the 10-nm sensing range of a single NV center brings us closer to performing EPR and NMR spectroscopy on individual-intact biomolecules. Magnetic resonance spectroscopy with single-molecule sensitivity could provide insights into various in vivo or in vitro applications, e.g., receptor-ligand binding events (e.g., pharmaceuticals or toxins), posttranslational protein modification (e.g., phosphorylation processes), the detection of subtle protein conformational changes in living cells, and the like. Such applications could enhance our understanding of complex signaling pathways that are not accessible by current technologies. Additional details follow.

### Example 2: Functionalization Using Al₂O₃ Adhesion Layer

Diamond-based sensing can be impacted by the thickness of any functionalization layer, as well as its surface morphology and surface coverage. Described herein are various characteristics of the surface at each step of a non-limiting functionalization procedure.

Single-crystalline diamonds slabs (2×2×0.5 mm³, Element Six, electronic grade, Catalog No. 145-500-0385) were sonicated in acetone and isopropanol for 5 min each and dried with nitrogen gas before Al₂O₃ deposition. The deposition of the Al₂O₃ layer was carried out in an Ultratech/Cambridge Savannah ALD System by alternatingly delivering trimethylaluminum (TMA) and H₂O gases at 200° C., 20 cycles for 2 nm layer and 550 cycles for 50 nm Al₂O₃ thickness.

The diamonds were then soaked in 10 mM KOH for 10 s before being rinsed with a copious amount of DDI water (Milli-Q), and dried in an oven set to 80° C. Silanization was achieved using freshly prepared 3% N-[3-(trimethoxysilyl)propyl]ethylenediamine (CAS 1760-24-3, ACROS Organics, Catalog No. AC216531000) in anhydrous acetone (extra dry, ACROS Organics, Catalog No. AC326801000) at room temperature for 20 min. Upon completion, the surfaces were rinsed with acetone and DDI water and dried with nitrogen gas. For PEGylation, solutions of heterobifunctional PEG of various molecular weights (m.w.) were freshly prepared at ˜0.5 M concentration in 100-mM NaHCO₃ buffer with a final pH between 8.0 to 8.5. Specifically, heterobifunctional PEG molecules mPEG-SVA (average mw. 2,000 and 5,000) and biotinPEG-SVA (average m.w. 3,400 and 5,000) were purchased from Laysan Bio, azidoPEG-NHS (average m.w. 5,000, Catalog No. JKA5086) were purchased from Sigma-Aldrich, and mPEG9-NHS (m.w. 553.6, Catalog No. BP-22624) and biotinPEG8-NHS (m.w. 764.9 Catalog No. BP-22117) were purchased from BroadPharm. The diamonds were immersed in the PEG solutions and incubated for 1 to 2 h in the dark at room temperature, before being extensively washed with DDI water and dried with nitrogen gas.

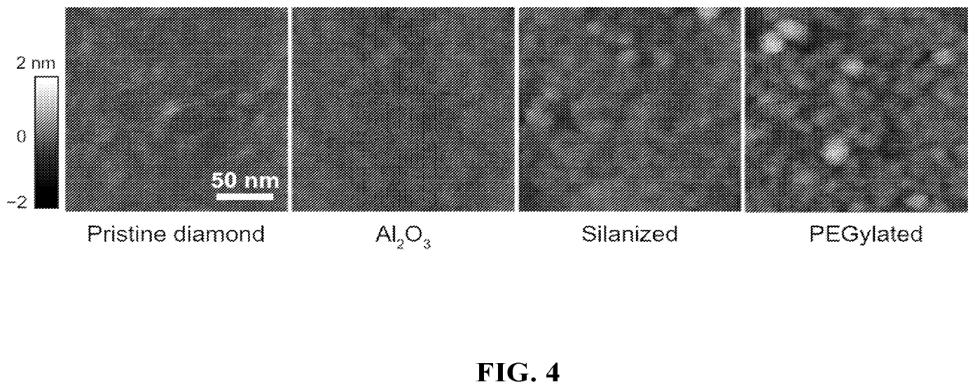

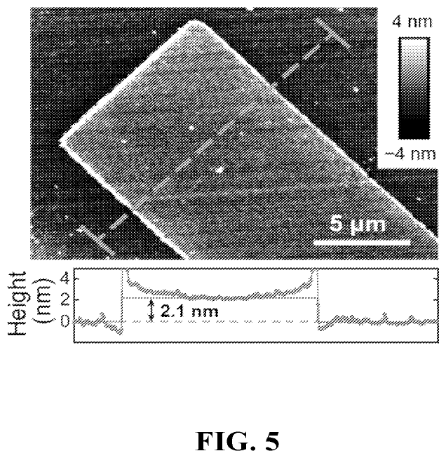

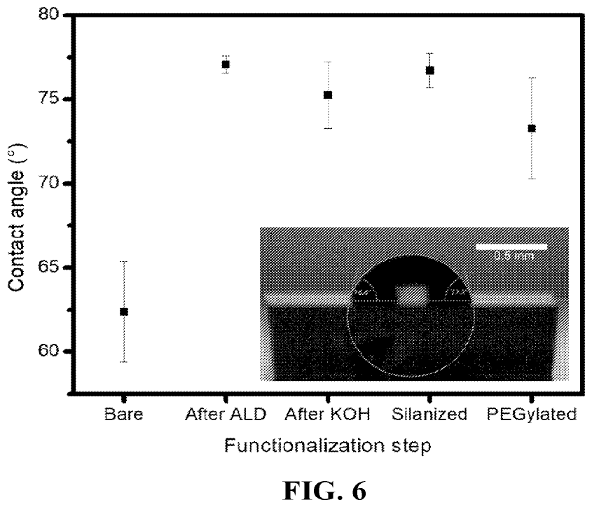

As confirmed by atomic force microscopy (AFM), thermal atomic layer deposition (ALD) resulted in deposition of a uniform, 2-nm-thick Al₂O₃ layer of excellent surface morphology (arithmetical mean deviation Rₐ=459 μm) on an oxygen-terminated diamond surface (Rₐ=446 μm) (FIG. 4, FIG. 5). The changes in surface properties were corroborated by contact angle measurement, as shown in FIG. 6. Contact angle measurements were performed with a Drop Analyzer Surface KRUSS DSA100 using a drop volume of 0.2 μL DI water. The contact angle was calculated based on the fitted curvature of each drop using the ellipse model in ADVANCE software. Prior to the measurements, diamond samples were rinsed with DI water, dried with N₂ gas flux, and then placed in an oven at 60° C. for one hour.

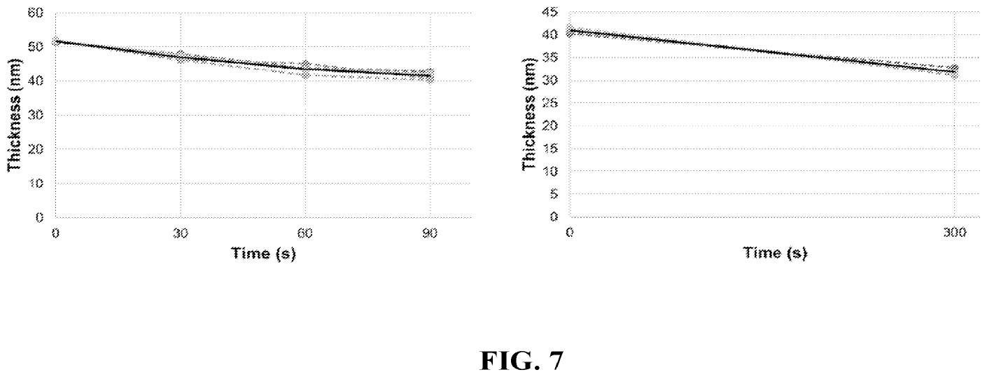

A slight increase of surface roughness was observed after treatment with 10 mM KOH for 10 s, which serves the purpose of —OH activation for silanization (Rₐ=841 μm) but also leads to hydrolysis of Al₂O₃, as shown in FIG. 7. For the data in FIG. 7, a 38-nm-thick Al₂O₃ film deposited with ALD on a diamond sample was patterned with a Heidelberg Direct Write Lithography system (AZ MiR 703 photoresist spin-coated 1 m and developed for 1 min in AZ 300MIF) and back etched (Cl₂/Ar in a Plasma-Therm ICP Etch system at 400 W ICP power for 55 s). Resulting diamond samples with lithographically patterned Al₂O₃ structures were submerged in 1 M KOH (FIG. 7, left) and 10 mM KOH (FIG. 7, right) for a fixed duration, rinsed immediately with water, and dried with N₂ gas. The dissolution rates in 1 M KOH and 10 mM KOH were 3.6 nm/min and 1.8 nm/min, respectively.

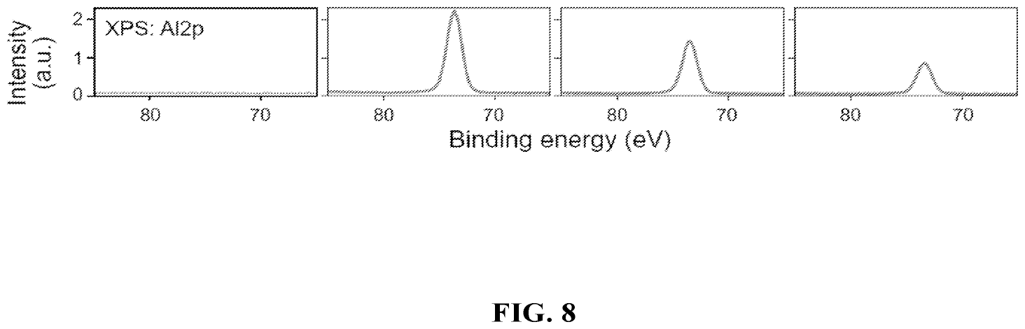

For the 2-nm-thick Al₂O₃ layer, a final surface roughness of Rₐ=866 μm was observed after PEGylation. X-ray photoelectron spectroscopy (XPS) further confirmed the presence of aluminum (especially the Al2p signal) after each surface treatment step, indicating that the Al₂O₃ layer remained stable during the processing (FIG. 8).

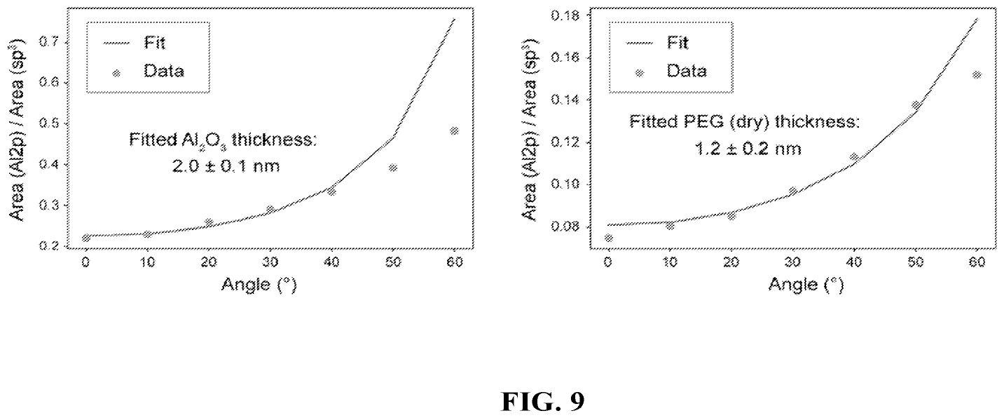

Using angle-resolved XPS (ARXPS), the thickness of the Al₂O₃ and PEG layer was further estimated to be 2.0±0 1 nm and 1.2±0.2 nm (FIG. 9). XPS was conducted using a Thermo Fisher K-Alpha and X-ray Spectrometer Tool with an Al K-alpha source (1486 eV) at Princeton University in the Imaging and Analysis center. Data were collected using a 250 μm spot size and a flood gun to mitigate charging. The angle between sample and detector was varied from 0-60°. Only 0° to 40° data were used for the fittings. The fitted results nicely followed the data except for at the higher angles, possibly owing to the pronounced elastic scattering under those conditions. Attenuation length values for photoelectrons passing through the different materials were taken from the NIST database.

For FIG. 9 (left), the Al₂O₃ layer was prepared by depositing 2 nm Al₂O₃ by thermal ALD (20 cycles of TMA+H₂O at 200° C.) onto a diamond sample. A 2-layer model (Al2p: only from Al₂O₃; C-sp³: only from diamond) as described in Lovchinsky et al., Science 351:836-41 (2016) was used to fit and extract the Al₂O₃ layer thickness. A density of 3.25 g/cm³ was used for amorphous Al₂O₃. The fitted thickness was 2.0±0.1 nm, which was in agreement with the expected value.

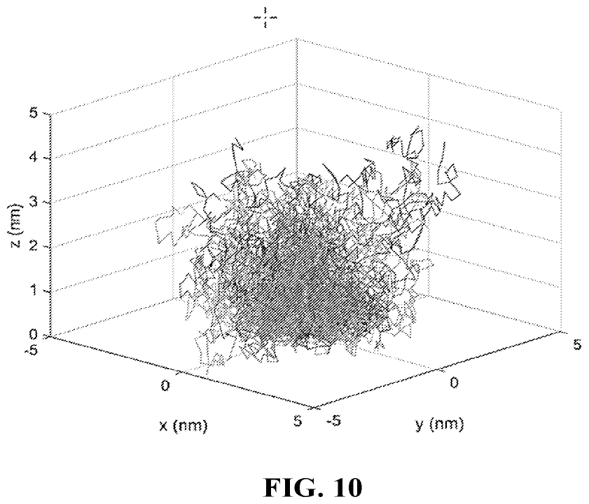

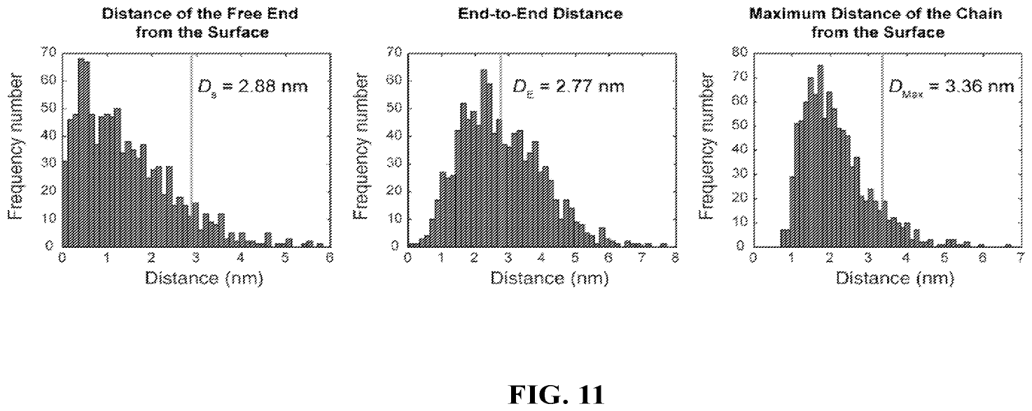

For FIG. 9 (right), the PEG layer was prepared on a diamond sample with 2 nm Al₂O₃ by silanization and PEGylation (using mPEG-2000 Da) as described above. A 3-layer model (Al2p: only from Al₂O₃; C-sp³: from both diamond and PEG layer) was used to fit and extract the PEG layer thickness. Reasonable fitting was achieved when the thickness of the intermediate Al₂O₃ layer was fixed to 1.0 nm, which was attributed to imperfections of the model. A PEG density of 0.3 g/cm³ was assumed, which was estimated from the volume occupied by each surface-bound PEG molecule (see FIG. 10, FIG. 11). The PEG layer thickness (vacuum-dried thickness of mPEG-2000 Da) was determined to be 1.2±0.2 nm. Note that the ARXPS data was collected under ultra-high vacuum and the PEG molecules may have collapsed. Errors for both thickness estimates are standard errors from the fits.

PEG molecules were modeled as random coil chains including N ethylene units with ethylene unit length of a. The reported value of a is between 0.27-0.42 nm under different assumptions and conditions. a=0.35 nm was used in this simulation. N was calculated from the average molecular weight, M, by N=M/44. Chains were generated in MatLab from the (0, 0, 0) origin with N number of connecting vectors, each of which had a fixed length of a and randomly oriented in a 3-dimensional space. No part of the chains was allowed to have negative z value, namely the chains were above the “surface” (x-y plane). Results are shown in FIG. 10 and FIG. 11. In addition, the PEG layer thickness was estimated based on the end-to-end distance d_{E} of a single PEG molecule, assuming a Gaussian chain behavior in a good solvent, namely dₑ=a\sqrt{N}. The estimated thicknesses of all PEG molecules based on the above-mentioned methods are summarized in Table 1.

TABLE 1

| | No. | d_{E} | D_{S} | D_{E} | D_{Max} |
|---|---|---|---|---|---|
| | units | (nm) | (nm) | (nm) | (nm) |
| PEG₈ | 8 | 0.99 | 0.89 | 0.85 | 1.00 |
| PEG₉ | 9 | 1.05 | 0.92 | 0.89 | 1.06 |
| PEG-2000 Da | 45 | 2.35 | 2.21 | 2.11 | 2.53 |
| PEG-3400 Da | 77 | 3.07 | 2.88 | 2.77 | 3.36 |
| PEG-5000 Da | 114 | 3.73 | 3.54 | 3.45 | 4.14 |

Dₛ is the 90^{th} percentile of the distance of the free end from the surface, D_{E} is the mean value of the end-to-end distance, and D_{Max} is the 90th percentile of the maximum distance of any part of each chain from the surface, as determined by the data analysis described above. In Table 1, D_{S}, D_{E}, and D_{Max} are metrics from analyzing 1000 simulated surface-anchored chains.

ARXPS likely underestimated the true thickness of the PEG layer, since ARXPS was performed under ultra-high vacuum, which could lead to a collapse of the PEG layer. Assuming a Gaussian chain model, the thickness of the hydrated PEG layer was estimated to be 2.8 nm (see FIG. 10, FIG. 11, Table 1). From this, the total thickness of the functionalization layer was estimated to be on the order of 5 nm.

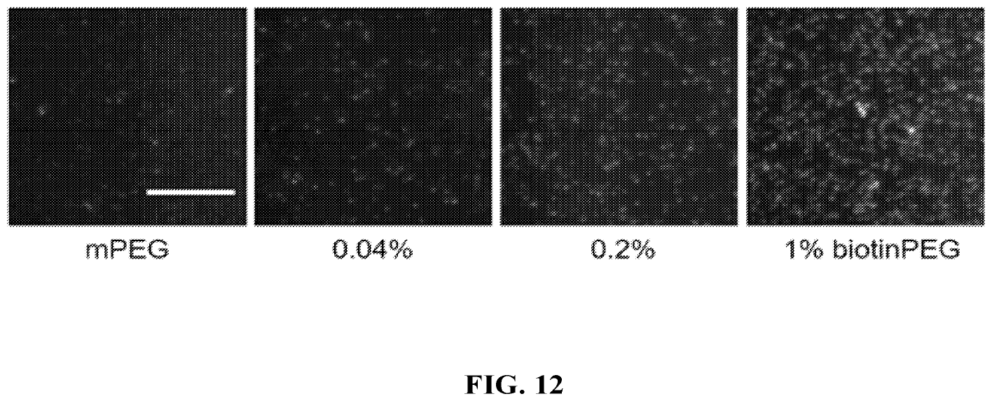

Notably, shorter PEG could be employed to further reduce the overall thickness to 3 nm without impeding the fine control over the grafting density, as shown in FIG. 12. Surfaces were functionalized with mPEG₈ and biotinPEG₉, which led to a PEG thickness of approximately 1 nm (see Table 1). Thus, the overall thickness of the functionalization architecture achieved was roughly 3 nm (2 nm Al₂O₃+1 nm PEG).

### Example 3: Single-Molecule Imaging and Bioconjugation

Described herein are non-limiting methods for controlling and characterizing the adsorption density of proteins on a diamond surface.

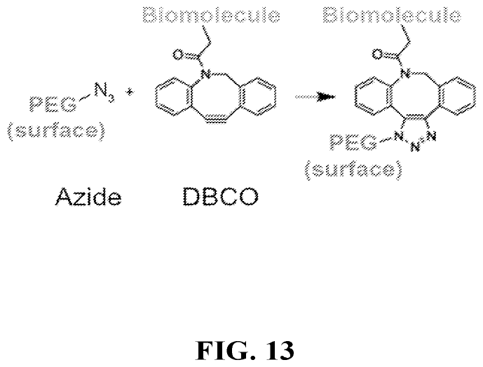

The density of binding sites was controlled by adjusting the stoichiometric ratio of methyl-terminated PEG (mPEG) and functional PEG groups, for example, biotin-terminated PEG (biotinPEG) or azide-terminated PEG (azidePEG) for click chemistry (FIG. 13). The adsorption density of Alexa 488 dye-labeled streptavidin (SA-488) on surfaces prepared according to Example 2 was characterized by single-molecule fluorescence microscopy.

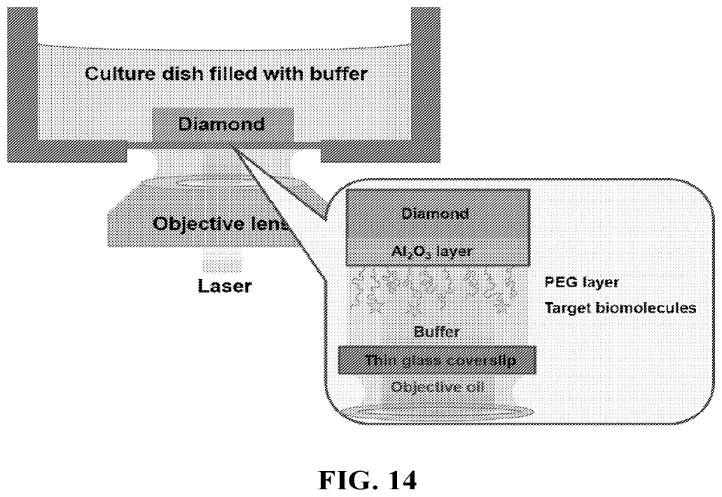

Single-molecule fluorescence imaging was performed on a custom-built fluorescence microscope equipped with 488- and 532-nm lasers (Coherent Sapphire) and a 60× oil objective (Olympus UPLAPO60XOHR) in an inverted configuration with epi-illumination. Diamond samples were placed inside a buffer-containing dish with the functionalized side facing down and imaged through the glass coverslip bottom that was also passivated with mPEG to minimize nonspecific binding (FIG. 14). As indicated in each case, samples were imaged in Invitrogen SlowFade Diamond Antifade Mountant (refractive index 1.42) instead of buffer for improved sensitivity and photostability. For both single-molecule fluorescence imaging and NV confocal imaging, Olympus Type F immersion oil (n=1.518) was used.

For 488-nm excitation (SA-488), a ZET488/10× (Chroma) notch filter, a ZT488rdc-UF1 dichroic beamsplitter, and an ET525/50 m emission filter were used. For 532-nm excitation (Cy3-ssDNA), a ZET532/10× notch filter, a ZT532rdc-UF1 dichroic beamsplitter, and an ET575/50 m emission filter were used. Images were acquired by an Andor iXon Ultra 888 electron-multiplying charge-coupled device (EMCCD) camera (EMCCD cooled down to −60° C.) with 1 s exposure time and 200 (or 300) gain, or 500×120 ms for video.

In some instances, the surfaces were reset prior to imaging experiments or spin coherent measurements. To reset a surface, diamonds can be first soaked in 1 M KOH (typically overnight but can be shortened), which effectively removes Al₂O₃ at a rate of 3.6 nm/min (FIG. 7). They were then immersed in NanoStrip at room temperature for 5 min, rinsed extensively with DDI water, sonicated in acetone and 2-isopropanol for 5 min each, and finally dried with nitrogen gas. This cleaning procedure proved to be reliable to restore the diamond for single-molecule fluorescence imaging experiments. For spin coherent measurements, while the above-mentioned regenerating procedure may be sufficient, the NanoStrip treatment was replaced by triacid cleaning, which uses a 1:1:1 mixture of nitric acid, perchloric acid, and sulfuric acid at boiling temperatures to ensure minimal contamination.

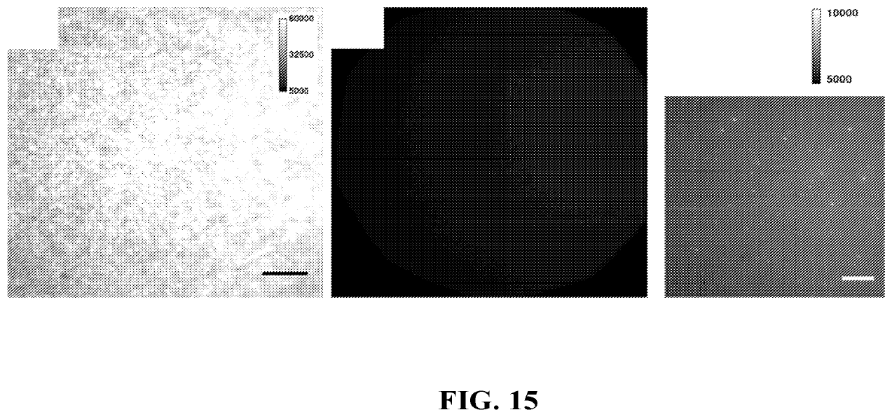

A silanized surface (amine-terminated, before PEGylation) and an mPEG-passivated surface, each prepared according to Example 2 on glass coverslips coated with 2-nm Al₂O₃, were incubated in SA-488 for 20 min, and then imaged by fluorescence microscopy. As shown in FIG. 15, massive non-specific binding can be seen on the silanized surface (left), in sharp contrast to the mPEG-passivated surface (center and right, displayed at a lower range).

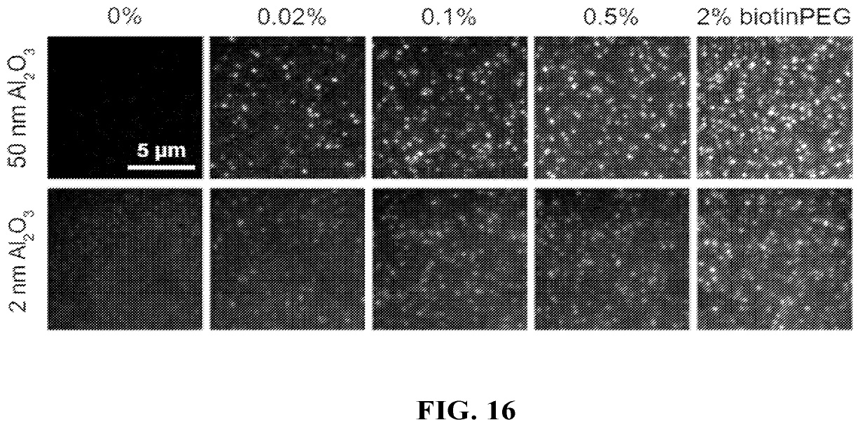

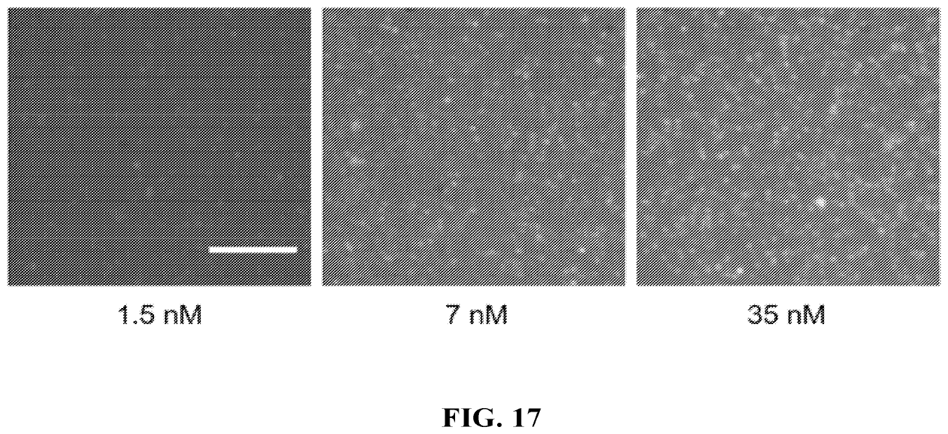

FIG. 16 shows a series of fluorescence images for diamond samples with varying biotinPEG density that were incubated in 7 nM SA-488 for 20 min. FIG. 17 shows a series of fluorescence images for diamond samples functionalized with 1% biotinPEG that were incubated at varied concentrations of SA-488.

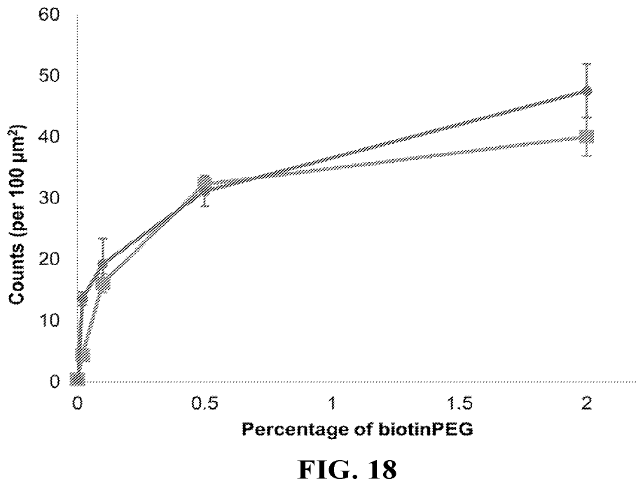

The number of fluorescence spots (i.e., individual streptavidin molecules) demonstrated a clear dependence of the adsorption density on the biotinPEG percentage. Notably, for the diamonds coated solely with mPEG, only 4×10⁻³ SA-488 protein per square micrometer was observed, whereas for 2% biotin, roughly 0.5 SA-488 per square micrometer was observed, as shown in FIG. 18. This suggested that the protein adsorption density could be controlled over more than two orders of magnitude simply by changing the PEG composition. For higher biotinPEG densities, individual SA-488 molecules were no longer optically resolvable.

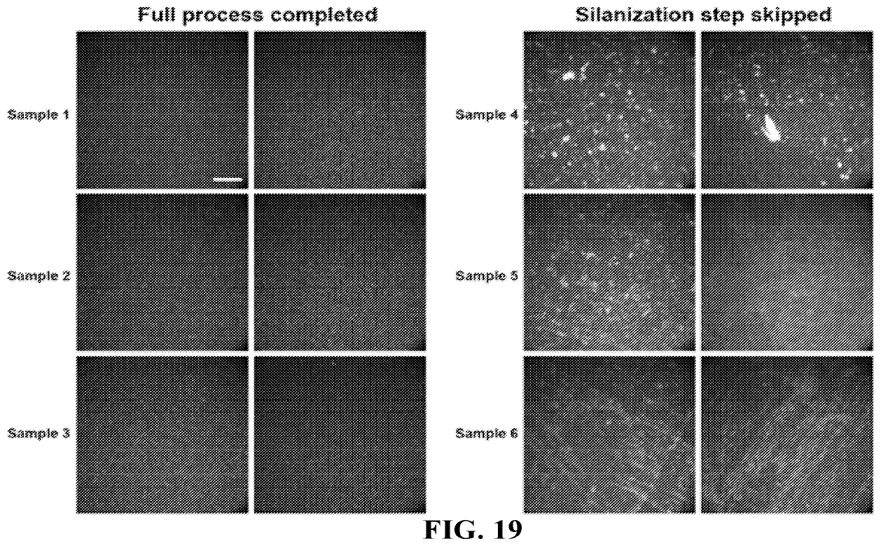

Furthermore, the immobilization of SA-488 was spatially homogeneous and highly reproducible, in sharp contrast to control experiments that skipped the silanization step, which resulted in a heterogeneous distribution of SA-488 on the diamond surface. As shown in the left column of FIG. 19, samples (50 nm Al₂O₃ thickness) that completed all functionalization steps were remarkably homogeneous with respect to the spatial distribution of immobilized streptavidin molecules. In contrast, as shown in the right column of FIG. 19, various forms of inhomogeneity were observed in samples prepared without a silanization step.

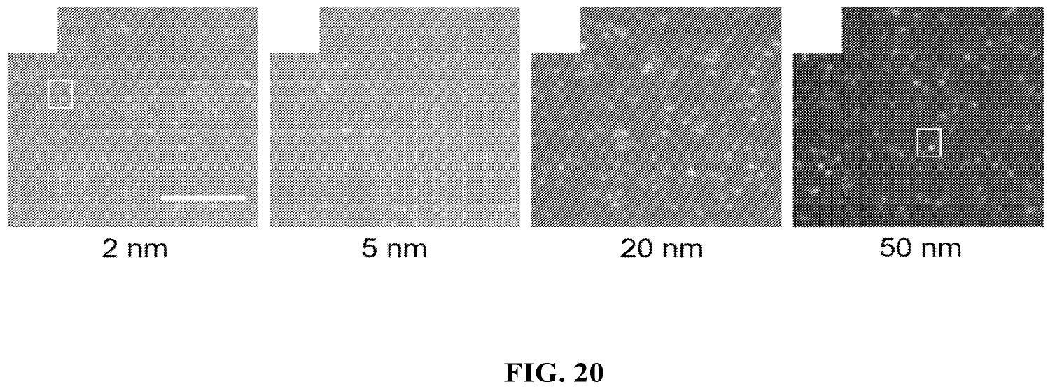

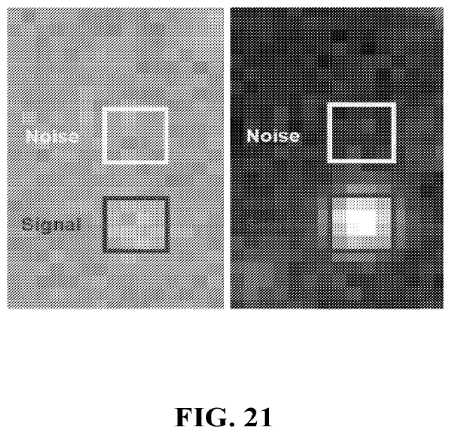

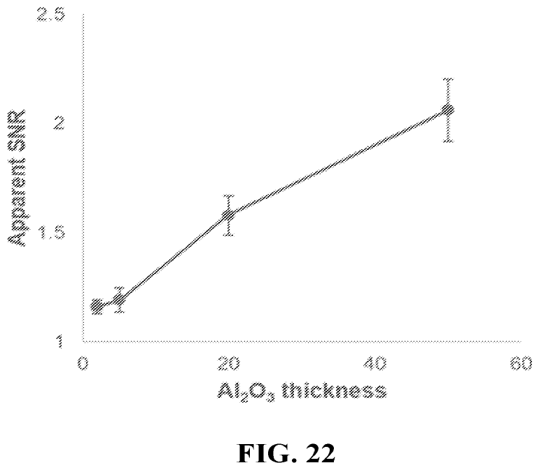

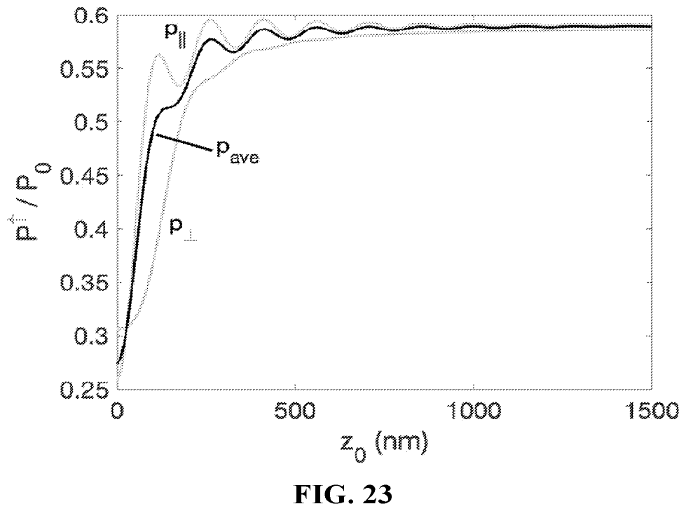

Performing these titration series for two different Al₂O₃ layer thickness, no qualitative difference in SA-488 adsorption density were observed between a 2-nm and a 50-nm Al₂O₃ layer, indicating that working with an ultrathin Al₂O₃ layer did not negatively impact biofunctionalization. Interestingly, a reduction in the signal-to-noise ratio (SNR) in fluorescence microscopy images was observed. As shown in FIG. 20 and FIG. 21, a clear reduction of the fluorophore SNR was observed as the Al₂O₃ layer thickness decreases. The sum of intensities of a 4×4-pixel area over a fluorescent spot (lower boxes in FIG. 21) were defined as the “signal,” and the sum of intensities of a 4×4-pixel area over a nearby background region (upper boxes) were defined as the “noise,” from which SNR was calculated. FIG. 22 shows the mean of apparent SNR quantified accordingly, based on 10 clearly distinguishable fluorescent spots in each image. At least partially, this observation could be explained by self-interference of an emitter at the diamond-Al₂O₃ interface. FIG. 23 compares the optical power emitted away from the diamond surface (PT) to the total power radiated (P₀) into all directions, including surface guiding modes. These calculations were based on an analytical solution (Keller, Principles of nano-optics principles of nano-optics, Lukas Novotny and Bert Hecht, Cambridge U. Press, New York, 2006. (2007)) for a dipole separated a distance (z₀) from a diamond-Al₂O₃ interface. The line labeled p_{∥} indicates the emitted power for a dipole parallel to the diamond surface, while the line labeled p_{⊥} denotes a dipole perpendicular to the surface. The black line (pₐᵥₑ) shows the emitted power for an isotropic molecule orientation. The fluorophore emission spectrum was extracted from fpbase.org/spectra/. Because of this, for single-molecule imaging experiments on samples that were coated with thin Al₂O₃ layers, Invitrogen SlowFade Diamond Antifade Mountant (refractive index 1.42) was used instead of water (refractive index 1.33) to improve SNR owing to better refractive index match and enhanced dye photostability.

The developed diamond surface modification architecture was readily combined with biochemical conjugation techniques. The versatility of this approach was demonstrated with two examples of bioconjugation: first, a molecular biological conjugation of target molecules to the diamond surface via biotin-streptavidin interaction and, second, a biochemical conjugation via “click chemistry.” For both examples, a Cy3 dye-labeled, 40-nt, single-stranded DNA (Cy3-ssDNA) served as a model molecule.

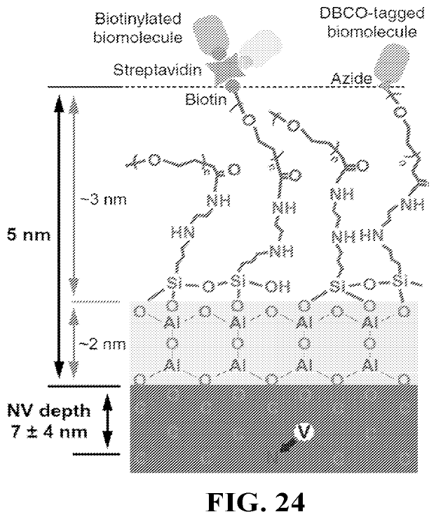

To immobilize biomolecules, 3 μL of 7-nM SA-488 (in 50 mM sodium phosphate buffer (pH 7.4) that also contained 100 mM NaCl) or 50 nM DBCO-tagged Cy3-ssDNA (in 50 mM sodium phosphate buffer (pH 7.4) that also contained 100 mM NaCl, and 1 mM MgSO₄) was carefully cast on the functionalized surface of each diamond and incubated at room temperature in a dark, moisturized environment for 20 min. For the streptavidin-mediated system, 20 nM biotin-tagged Cy3-labeled ssDNA was premixed with 40 nM nSA at 1:1 volume ratio and incubated at room temperature for 20 min with mild agitation, which was expected to result in 1-ssDNA:1-streptavidin conjugates with an effective concentration of 10 nM. This solution was then applied to diamond surfaces in the same way. Upon completion, the diamond was gently rinsed with the same buffer and placed in an imaging dish for fluorescence microscopy study. The primary sequence of the 40-nt ssDNA was 5′-TTTTT TTTTT AGTCC GTGGT AGGGC AGGTT GGGGT GACTT-3′ (SEQ ID NO:1). The biotin-tag (or DBCO-tag), followed by a Cy3-label, was attached to the 5′ end of the ssDNA. The biotin-based and azide-based systems are illustrated in FIG. 24.

In a typical experiment, a movie of the immobilized sample comprising 500 ms×100 (or 120) frames of images that have 512×512 pixels was recorded. Each movie was converted to a 16-bit TIF file. Based on the 1^{st} frame, individual fluorescence spots were identified using ImageJ by setting appropriate intensity cut-off and their rough positions were saved as a peak list. For each fluorescent spot, a 9×9-pixel square was created around its rough position, and a Gaussian fit was performed (only on the 1^{st} frame) to find out the accurate position as well as the standard deviation s. To extract intensity from each frame, the data values of all the pixels in a squared region with edge length of s (typically 3 or 4 pixels) centered at the identified accurate position were summed, subtracting the normalized background intensity defined by the residual region in the 9×9-pixel square. The final intensity accounted for file conversion and is reported as photon counts per second (s⁻¹). Typically, only the center 256×256-pixel region of each movie was analyzed, and fluorescence spots that were not completely photon-bleached by the end were eliminated for subsequent ensemble analysis.

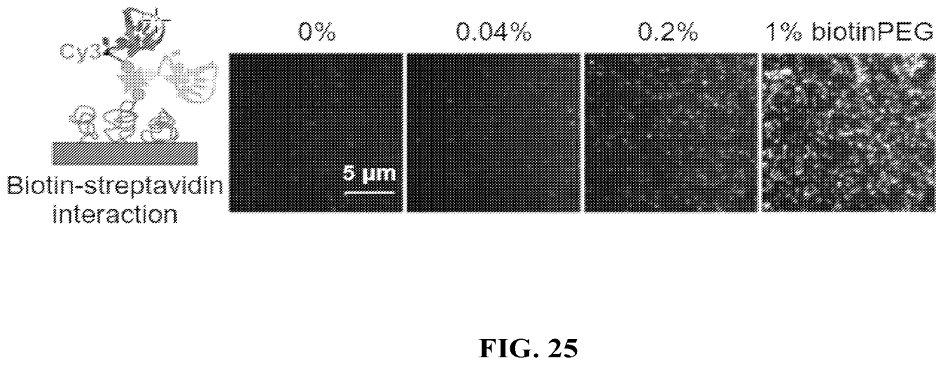

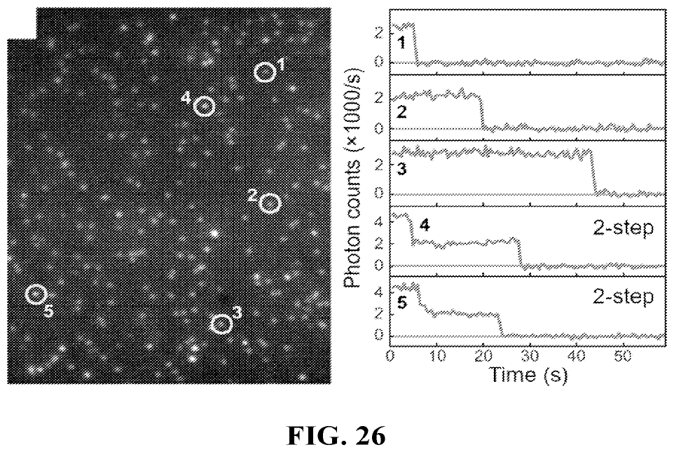

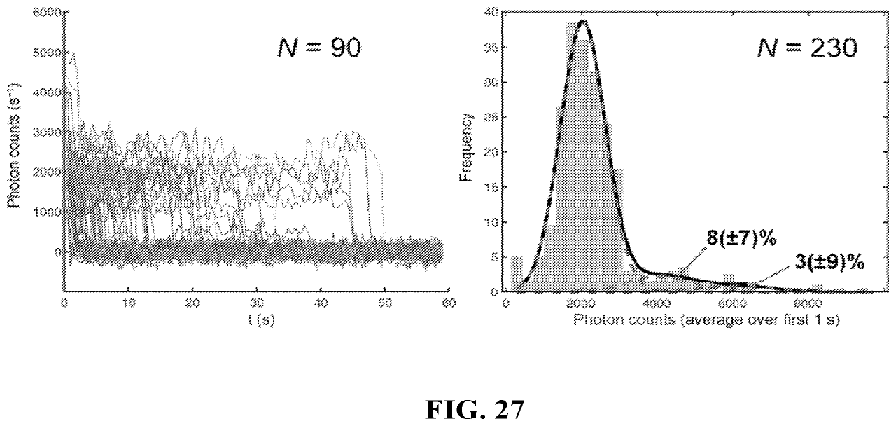

In the first system (FIG. 25), biotinylated Cy3-ssDNA was immobilized to the diamond surface mediated by streptavidin with no fluorescent label (nSA). Time-dependent fluorescence measurements show a single-step decay in the fluorescence signal for the majority of fluorescence spots, a hallmark for single-molecule measurements (FIG. 26). FIG. 27 shows (left) a plot of 90 fluorescence intensity traces extracted from a 256×256-pixel region of FIG. 26. Most of the traces had similar plateau values around 2000 counts/s and several traces displayed two-step decay behavior, as shown in FIG. 26. FIG. 27 also shows (right) a histogram of the average intensities over the first second (first 2 frames) from in total 230 traces based on three movies, decomposed to three Gaussian distributions that had equally spaced centers and the same standard deviation. From this, the population of biotin-DNA: nSA=[1:1, 2:1, and 3:1] complexes was estimated to be 89%, 8(±7)%, and 3(±9)% respectively (uncertainties were based on 95% confidence interval from the fit and only reported for small percentages). In addition, two-step decay events were also observed, which could be explained by multiple ssDNA molecules binding to a single nSA homotetramer, or coincidental colocalization of two nSA molecules.

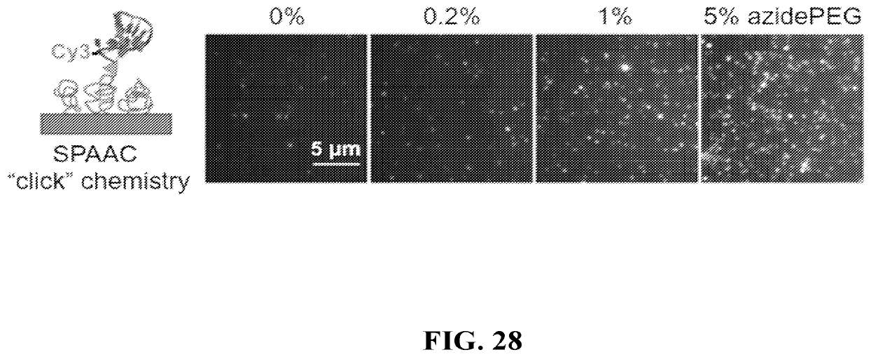

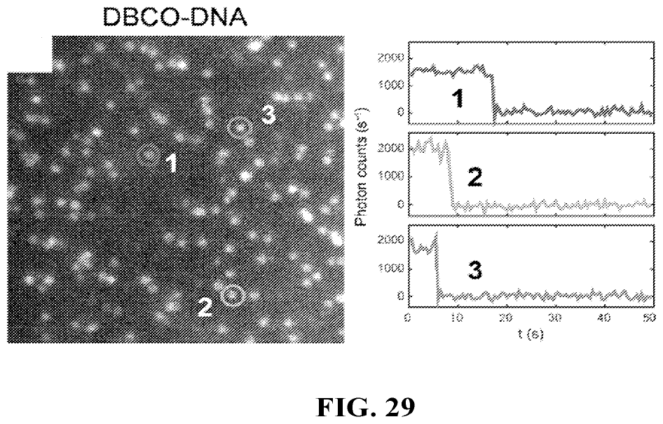

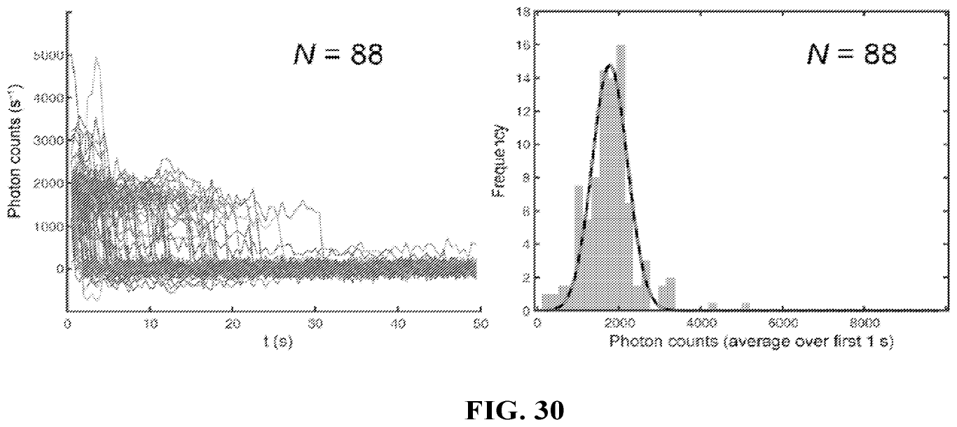

The second system (FIG. 28) exploited strain-promoted azide-alkyne cycloaddition (SPAAC), also known as “copper-free click chemistry” (FIG. 13), for its reliable performance and fast kinetics. The diamond surfaces were prepared following the same procedure described above, except that biotinPEG was replaced by an azide-PEG compound. Through SPAAC, the same Cy3-ssDNA engineered with a dibenzocyclooctyne (DBCO) label at its 5′ was successfully immobilized to diamond surfaces (FIG. 28 and FIG. 29). The large fluorescence spots, which included more than one fluorophore, likely originated from the presence of aggregates in the DBCO-tagged Cy3-ssDNA sample. FIG. 30 shows (left) a plot of 88 fluorescence intensity traces extracted from a 256×256-pixel region of FIG. 29. FIG. 30 also shows (right) a histogram of the average intensities over the first second (first 2 frames) from these 88 traces, fitted to a single Gaussian distribution.

### Example 4: Stability Under Physiological Conditions

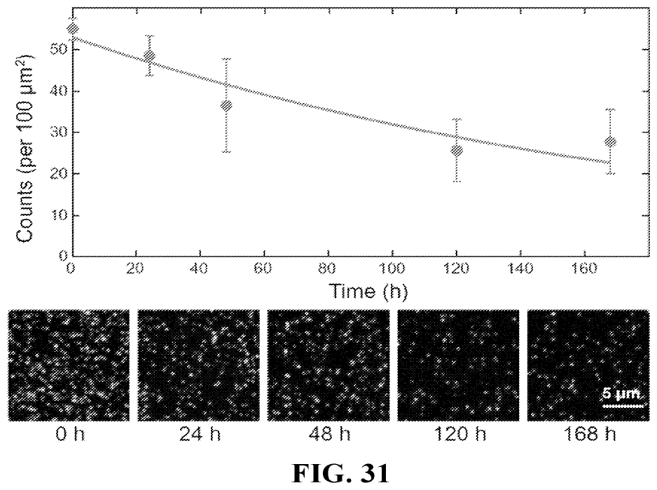

For any practical biophysical or diagnostics applications, it would be useful for the functionalization layer to maintain good chemical stability without degrading over the course of a typical experiment. Oxides can hydrolyze over time when exposed to water, at a rate that depends on the film (e.g., composition, deposition method, and film quality) and solvent (e.g., pH and salinity) properties. The chemical stability of the functionalization architecture of Examples 2 and 3 was tested. SA-488 was immobilized to a diamond surface, and the fluorescence thereof was monitored over the course of a week while storing the sample in a sodium phosphate buffer (pH 7.4, [NaH₂PO₄+Na₂HPO₄]=50 mM, [NaCl]=100 mM) at 23.5° C. (room temperature). FIG. 31 shows the observed fluorescence signal over time for a surface including a 50-nm-thick Al₂O₃ layer. The decrease in the number of SA-488 per field-of-view could be attributed to either a dissociation of the functionalization layer or photobleaching of the SA-488. This set an upper limit for the functional layer dissociation rate to a half-life time of 5.7 days (d).

In addition to these optical measurements, AFM was also used to monitor changes of the Al₂O₃ layer thickness as a function of submersion time in doubly deionized (DDI) water and sodium phosphate buffer. AFM was performed on a Bruker Dimension Icon instrument with SCANASYST-AIR tips. Images were flattened to the 1^{st} order using NanoScope Analysis software.

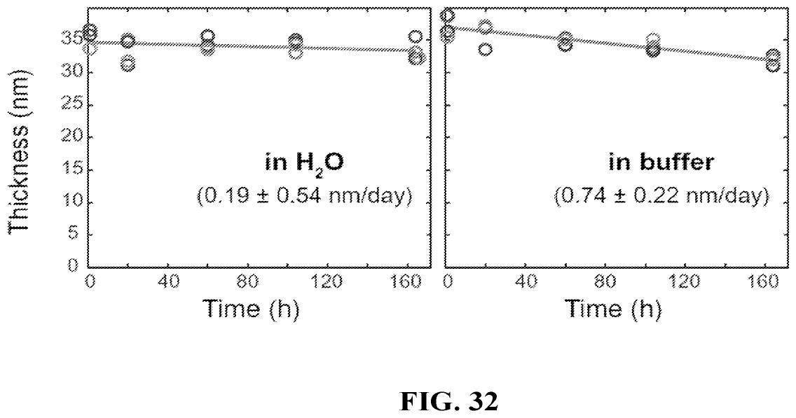

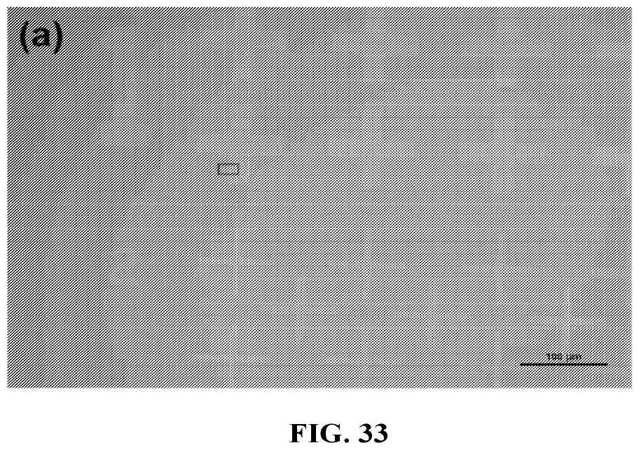

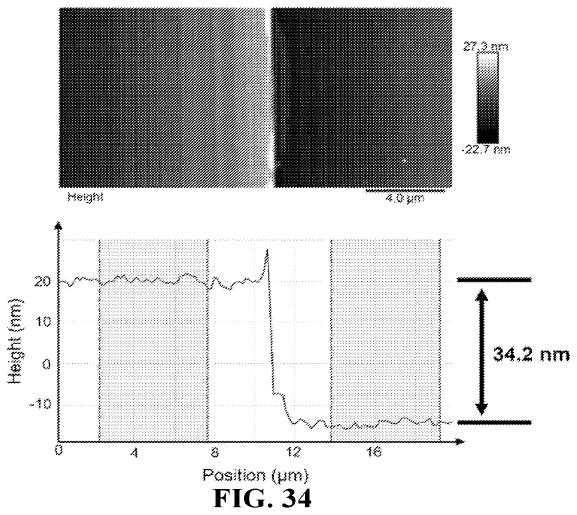

FIG. 32 shows the thickness of a lithographically patterned Al₂O₃ structure as a function of submersion time measured by AFM. Examples of measurements of the remaining thickness of the functional layer are shown in FIG. 33 and FIG. 34. FIG. 33 is an optical image of a diamond chip with lithographically patterned Al₂O₃ layer that was also silanized and PEGylated according to Example 2. Cross-shapes were where Al₂O₃ had been removed by reactive ion etching. FIG. 34 (top) is an AFM scan (20×10 μm², 128×64 lines) of the region indicated by a box in FIG. 33. FIG. 34 (bottom) is a plot of the 2D profile of FIG. 34 (top) 33 averaged along the short edge. The profile has been leveled (1st order) to correct the baseline. The height difference can thereafter be extracted based on the two color-shaded regions, which reflects the thickness of the remaining functional layer in respect to diamond substrate.

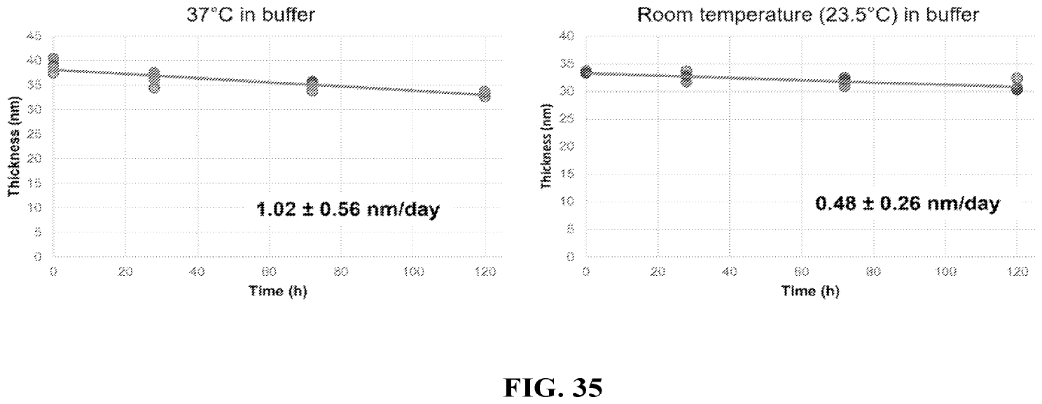

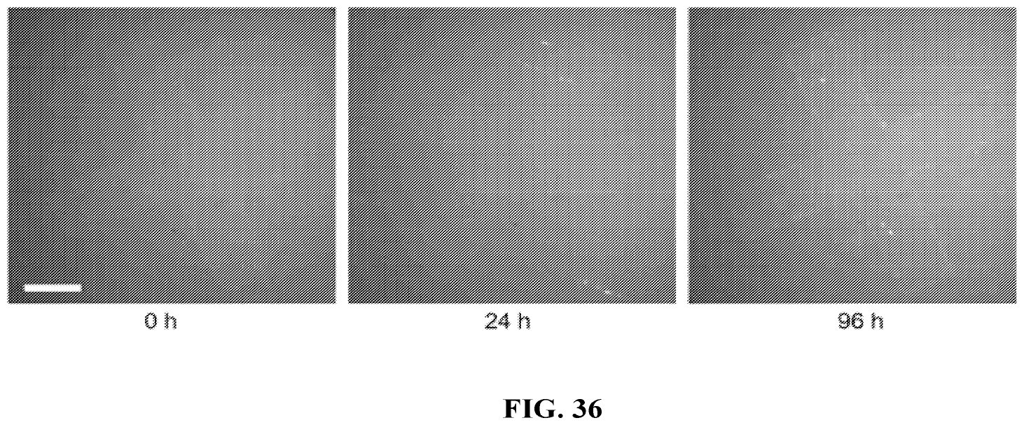

In water, dissociation was negligible (observed rate 0.19±0.54 nm/d with a large uncertainty), which was in good agreement with the optical measurements. However, for the measurements in sodium phosphate buffer, a dissociation rate of 0.74±0.22 nm/d (and 1.02±0.56 nm/d at 37° C., as shown in FIG. 35) was determined, which is slightly larger than what would be expected from the optical measurements in FIG. 31 and from the direct optical observation of the lithographic Al₂O₃ patterns shown in FIG. 36. In FIG. 36, the “cross” shaped Al₂O₃ patterns (2 nm thickness) were lithographically written on a diamond sample and then immersed in 50 nM sodium phosphate buffer (pH 7.4, also containing 100 mM NaCl) at room temperature, over a course of 4 days. The pattern was created by a lift-off (for 2 nm thin layer) process. Specifically, diamond was patterned with AZ MiR 703 photoresist on a Heidelberg Direct Write Lithography system with 375 nm laser exposure, then deposited with 2 nm thick Al₂O₃ (20 cycle, 200° C., the photoresist color turned red at this temperature, but patterns were unperturbed), and finally lifted off in 80° C. NMP for 40 min under sonication.

In the images of FIG. 36 (488 nm laser excitation), the Al₂O₃ regions were slightly brighter, possibly due to the presence of fluorescent crystal defects in the ALD Al₂O₃. Such contrast disappeared rapidly during short exposure (˜5 s) to the ˜30 mW 488 nm excitation laser, making the attempt to directly compare the remaining thickness (deduced from the contrast) between images almost impossible. Certain patterns (especially the rightmost one) exhibited extraordinarily bright edges, which likely originated from remaining photoresist due to incomplete lift-off during lithography, as shown in FIG. 5. Notably, no visible damage, including pinholes or pill-off of edges, to these patterns were observed during the entire period, suggesting that the Al₂O₃ thin layer was stable under (near-)physiological conditions.

Without wishing to be limited by mechanism, the observed optical and AFM results suggested that the Al₂O₃ layer on diamond undergoes a continuous dissolution process, rather than a sudden detachment.

### Example 5: Qubit Coherence

The electronic spin of individual NV centers was initialized and read out using a 520-nm green laser (Labs-Electronics, DLnsec). The spins were coherently manipulated by a microwave signal generator (Stanford Research Systems, SG 396) with a build-in in-phase and quadrature (IQ) modulator. The microwave pulse phase and length were controlled by an arbitrary waveform generator (Zurich Instrument, HDAWG8-ME) via IQ modulation. The modulated microwave was then amplified with a high-power amplifier (Mini-Circuits ZHL-16W-43+) and delivered via a coplanar waveguide to the diamond sample. A home-built confocal microscope was used to collect NV fluorescence, which was equipped with a dichroic beam splitter (Chroma, T610lpxr) to separate excitation and emission pathways. The emission was detected by a single-photon counter (Excelitas SPCM-AQRH-14) and processed by a time tagger (Swabian Instruments, Time Tagger 20). Confocal scanning was achieved by a piezo scanner (Mad City Lab, NANOM 350). External magnetic fields were provided by a neodymium-permanent magnet (K&J Magnet), which was mounted on a motorized translation stage with four degrees of freedom (Zaber technologies) for full control over the magnetic field alignment. The involved devices were synchronized and triggered by a transistor-transistor logic pulse generator (Swabian Instruments, Pulse Streamer 8/2).

The diamond sample used for NV spin coherence measurement was prepared following the procedure outlined in Sangtawesin et al., Phys. Rev. X9:031052 (2019). Briefly, a scaife-polished diamond sample (Element Six, Catalog No. 145-500-0385) was etched with Ar/Cl₂ followed by O₂ plasma to remove the top few micrometers of material. The top few nanometers were then intentionally graphitized by annealing to 1,200° C. for 2 h in a vacuum tube furnace. The diamond was then implanted with ¹⁵N by Innovion Corporation (3 keV, 3×10⁹/cm², 0° tilt) and subsequently annealed to 800° C. in a vacuum tube furnace to form NV centers, followed by oxygen annealing at 460° C. Coherence measurements before and after surface modification were performed at 1,750 G magnetic field strength. Both YY-8 and spin-echo pulse sequences were used to measure the coherence and depth of NV centers.

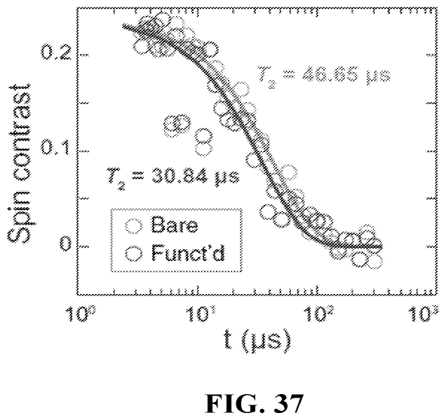

The normalized signal (spin contrast) is defined by 2[F₀(t)−F₁(t)]/F₀(t)+F₁(t)], which removes the common-mode noise [F₀(t) refers to the detected fluorescence in |0 [symbol] and F₁(t) to that in |−1 [symbol] ]. The coherence data in FIG. 37 are fitted to a stretched exponential function exp[−(t/T₂)ⁿ]. T₂ and the number of π-pulse at which T₂ saturates are fitted to either a saturation curve.

$$\\ \begin{matrix}{T}_{2} ( N ) = {T}_{2} ( 1 ) [ {N}_{sat}^{{}_{} S} + ( {N}^{{}_{} S} - {N}_{sat}^{{}_{} S} ) exp ( - N / {N}_{sat} ) , & [ 1 ]\end{matrix}$$

or to a power law:

$$\\ \begin{matrix}{T}_{2} ( N ) = {T}_{2} ( 1 ) {N}^{{}_{} 2} , & [ 2 ]\end{matrix}$$

where no saturation of T₂ is observed. The final fitting parameters are given in Table 2.

TABLE 2

| | T₂ (μs) | s | Nₛₐₜ |
|---|---|---|---|
| NV No. 2 Bare | 7.04 | 0.40 | — |
| NV No. 2 Functionalized | 0.70 | 0.64 | — |
| NV No. 7 Bare | 23.70 | 0.44 | 130.082 |
| NV No. 7 Functionalized | 28.01 | 0.25 | — |

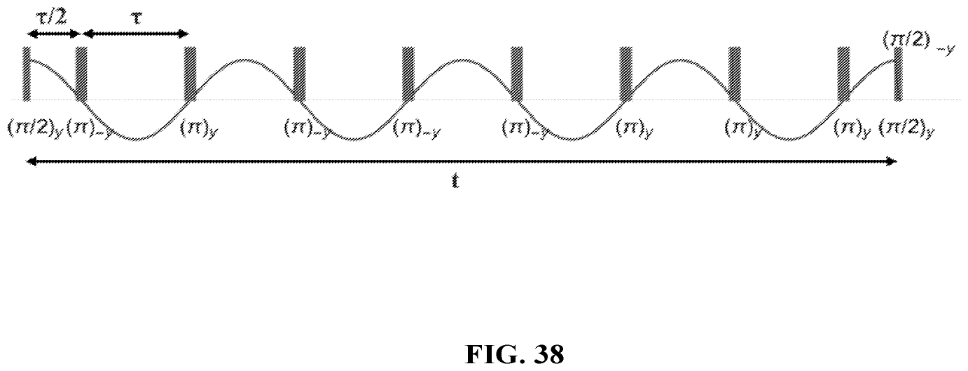

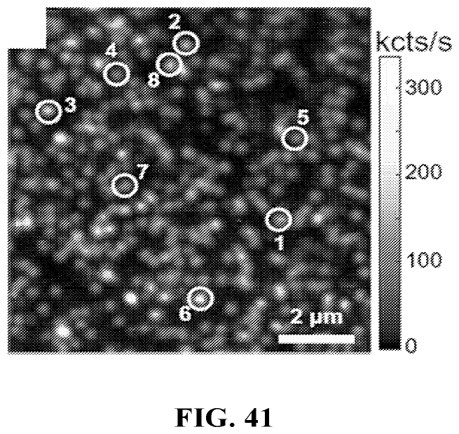

The impact of the functionalization architecture of Examples 2 and 3 on the spin coherence (T₂) of near-surface nitrogen vacancy (NV) centers was also studied. Long coherence times can affect NV-based quantum sensing because the sensitivity is generally proportional to ˜\sqrt{T₂}. FIG. 37 shows an example of the coherence time of an NV center under (YY-8)_{N=8} dynamical decoupling before and after surface modification for NV center number 3 (depth 4.8 nm) of FIG. 41. YY-8 sequences were chosen for their robustness to pulse errors and the ability to suppress spurious signals from nearby nuclear spins. An illustration of the YY8 pulse sequence is shown in FIG. 38. Eight w-pulses with either y or −y phase equally spaced by time τ are placed between two π/2-pulses. The last π/2-pulse has a 180° phase shift to cancel out common mode noise. The entire duration of the YY8 pulse is denoted by t, which is the x-value in FIG. 37. YY8 pulse sequences were used, which, compared to commonly used XY-type sequences, could effectively avoid the higher-order spurious signal due to pulse imperfection.

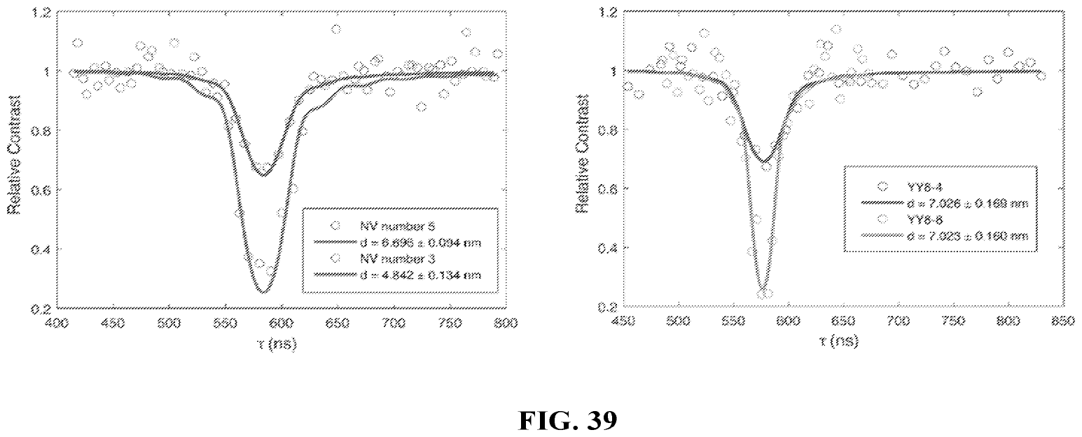

FIG. 39 (left) shows examples of the depth measurement, where two NV centers of different depths gave rise to clearly different relative contrast. As shown in FIG. 39 (right), depth measurements on the same NV center using a different number of t-pulses returned essentially the same results, which verified the robustness of the method. All measurements were performed at 200 G, which resulted in a Larmor precision frequency accessible by an XY-lock-in measurement technique.

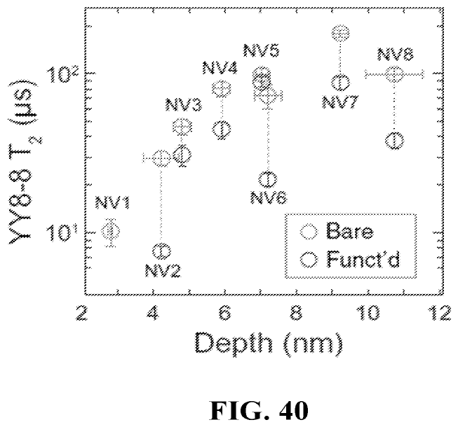

The observed coherence followed a stretched exponential with exp[−(t/T₂)ⁿ], with T₂=47 μs before and T₂=31 μs after the functionalization (the exponent n in FIG. 37 and FIG. 40 ranges from 1 to 1.8). T₂ and the longitudinal spin relaxation (T₁) times were further systematically investigated for eight spatially resolved NV centers with depths ranging from 2.3 to 11 nm (FIG. 40, for centers identified in FIG. 41), where the NV depths were determined by probing noise from the environmental ¹H spins in the immersion oil following the method described in Pham et al., Phys. Rev. B 93:045425 (2016).

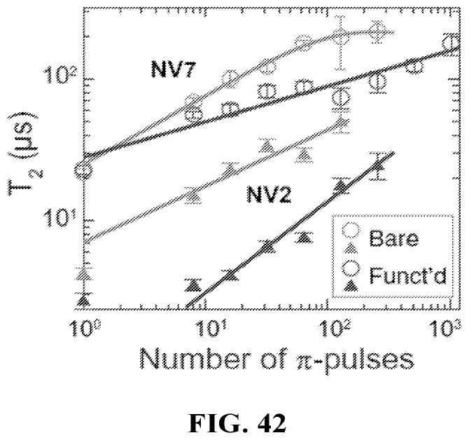

FIG. 42 shows T₂ times as a function of the number of R-pulses for NV number 2 (depth 4.2 nm, triangles) and NV number 7 (depth 9.2 nm, open circles) of FIG. 39, before (light gray) and after (dark gray) functionalization. Solid lines are fits based on Eqs. 1 and 2, above.

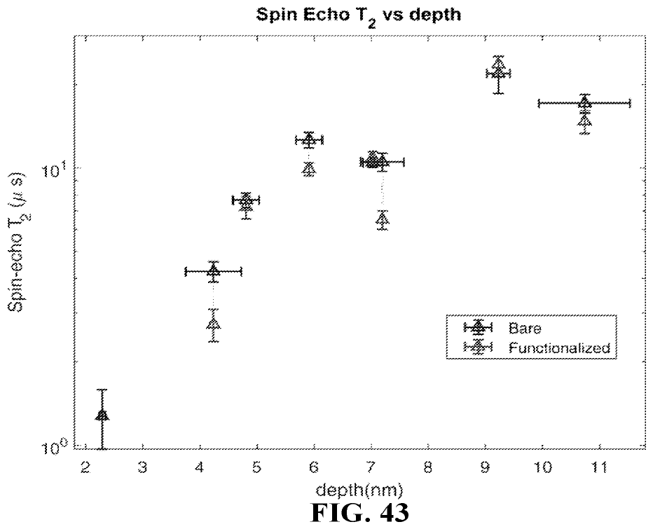

All investigated NV centers, with the exception of the shallowest NV (depth 2.3 nm), maintained their coherence after functionalization, with an observed characteristic increase in T₂ as a function of NV depth. Overall, the T₂ of these NVs upon surface functionalization was reduced by 49±22% under (YY-8)_{N=8}, or 15±18% when using a spin-echo sequence. FIG. 43 shows T₂ measured by a spin-echo pulse sequence plotted against NV depth before (dark gray) and after (light gray) functionalization for the same set of NV centers shown in FIG. 41. Analogously to the YY8 pulse in FIG. 40, a typical T₂ dependent on NV depth was seen. The fold-decreases in T₂ after functionalization measured by spin-echo was, however, much smaller than the ones revealed by YY8 sequences.

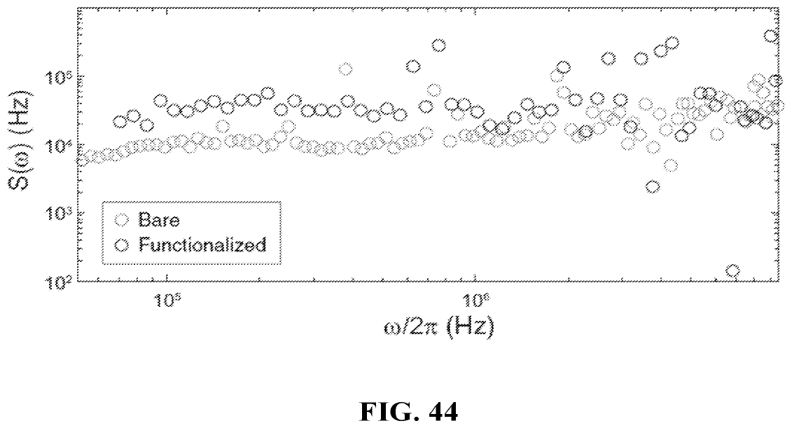

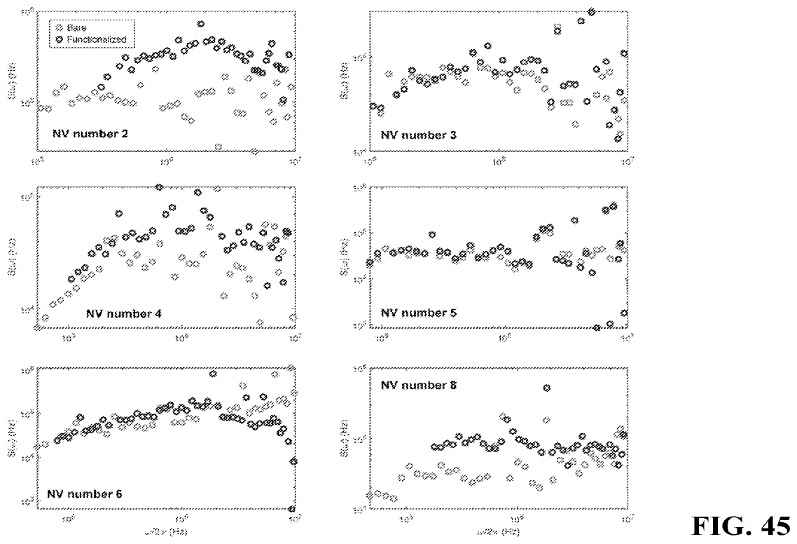

A careful investigation on spectral decomposition revealed a broadband noise spectrum across the frequency range of 0.05 to 10 MHz (FIG. 44 and FIG. 45). The spectral density of noise S(ω) was estimated from coherence measured by dynamical decoupling sequence. The measurement contrast could be written as C(t)=e^{-χ(t)} (

$$\\ χ ( t ) = \frac{1}{π} \int \frac{S ( ω )}{{ω}^{{}_{} 2}} 8 {sin}^{4} ( \frac{ω t}{4 N} ) \frac{{sin}^{2} ( \frac{ω t}{2} )}{{cos}^{2} ( \frac{ω t}{2 N} )} d ω$$

(note, N refers to the number of π-pulses)). For large N, where F_{N}(ωt) is sharply peaked at ω=πN/t, the equation can be approximated as

$$\\ χ ( t ) \approx \frac{tS ( ω )}{π} .$$

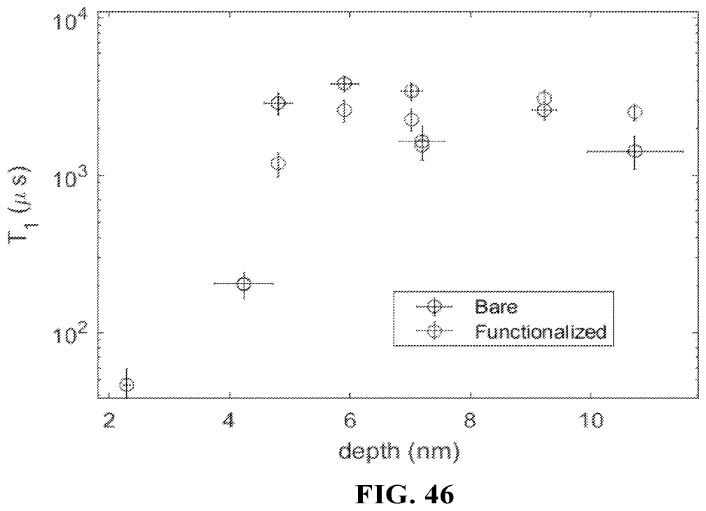

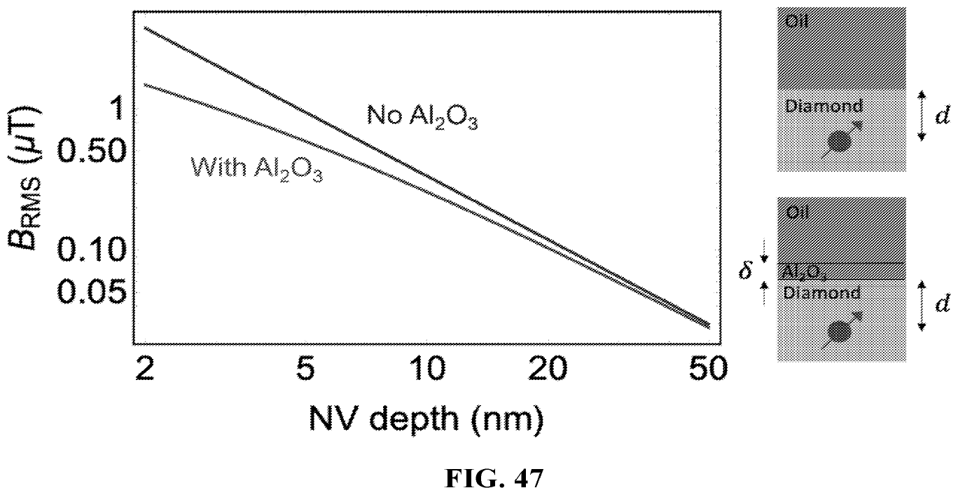

A sizable reduction in T₁ time (FIG. 46) was not registered after surface treatment, suggesting that charge and magnetic field noise spectra had neglectable frequency components at 3 GHz. The observed decrease in NV coherence could not be explained by the presence of an ²⁷Al nuclear spin bath (FIG. 47) but could be attributed to a noisy environment introduced by paramagnetic defects in the Al₂O₃ layer. FIG. 47 shows analytical results for the root-mean-square magnetic field noise (B_{RMS}) that is experienced by an NV center of depth (d) below the diamond surface. The curve labeled “No Al₂O₃” corresponds to a bare diamond, where the NV center only sees noise from ¹H spins in the oil (schematic on the top right shows oil, diamond and NV center (black)). The curve labeled “With Al₂O₃” corresponds to the case of a diamond coated with a 2 nm-thick Al₂O₃ layer. In this case, the NV center sees fields from both ²⁷Al and ¹H spins (schematic on the bottom right shows the same as on the top, but with an Al₂O₃ layer between the oil and the diamond). The analytical simulations are based on a model taken from Pham et al., Phys. Rev. B. 93 (2016) that has been generalized for multiple layers:

$$\\ {B}_{RMS}^{{}_{} 2} = {\sum }_{k} \frac{5 π}{96} {{ρ}_{k} ( \frac{{μ}_{0} ℏ {}_{} {γ}_{k}}{4 π} )}^{2} ( \frac{1}{{d}_{k}^{{}_{} 3}} - \frac{1}{{( {d}_{k} + {δ}_{k} )}^{3}} ) ,$$

where ρₖ denotes the nuclear spin density, γₖ denotes the nuclear spin gyromagnetic ratio, dₖ denotes the position, and δₖ denotes the thickness of the k^{th} layer. μ₀ denotes the vacuum permeability, and h denotes the reduced Planck constant.

Based on the experimental parameters in FIG. 37, the required integration time to detect a target nuclear spin was estimated. In NV sensing, the signal from an individual target spin is given by

$$\\ Signal = \frac{T}{τ + {t}_{read}} \frac{{f}_{0} - {f}_{1}}{2} χ ( τ ) \vert sin ( A τ / ℏ ) \vert ,$$

where T denotes the total measurement time, τ is the phase accumulation time, t_{read} is the optical spin-readout time, f₀=0.063 (f₁=0.048) is the average number of detected photons per readout window in mₛ=0 (mₛ=1), χ is the spin contrast from FIG. 37, A is the hyperfine coupling between the NV and the target spin, and ℏ is the reduced Planck constant. The noise (standard deviation) is given by

$$\\ Noise = \sqrt{\frac{T}{τ + {t}_{read}} \frac{{f}_{0} + {f}_{1}}{2}} .$$

The NV center in FIG. 37 has a depth of 4.8 nm, adding an additional 5 nm for the surface functionalization (i.e., 2 nm Al₂O₃ and the subsequent silanization and PEGylation using only mPEG). Taking into account that the diamond has a (1,0,0) cut, we estimated the average interaction strength between NV centers and ¹³C spins to be A=(2π) 160 Hz. Based on these parameters, the required integration time was estimated to be 2.8 hours. These demanding integration times could further be reduced to 100 seconds by utilizing quantum logic sequences (e.g., 100 repetitions).

Based on the demonstrated sensor-target distances and qubit coherence, the NMR signal of an individual ³C nuclear spin could be detected with integration times as short as 100 s. The anticipated integration time could further be reduced by minimizing the overall thickness of the functionalization layer and increasing the NV coherence time. A decrease in the functionalization layer thickness could be achieved by the deposition of a sub-1-nm Al₂O₃ layer and the passivation with shorter PEG, whereas the coherence time could be increased through further material processing, such as optimization of Al₂O₃ growth parameters and additional annealing after Al₂O₃ deposition, as well as increasing the number of z-pulses during dynamical decoupling.

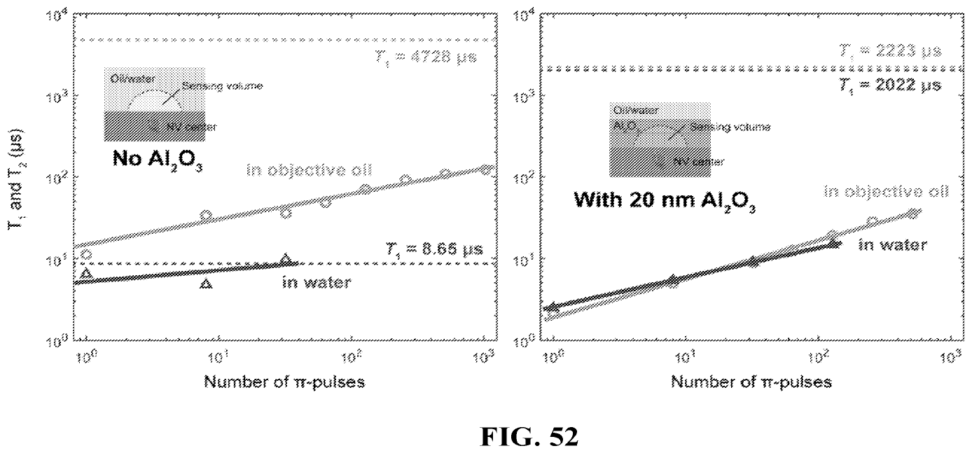

FIG. 52 shows the impact of a 20 nm-thick Al₂O₃ on T₁ and T₂ coherence times measured in objective oil and water (left) before and (right) after functionalization. For shallow NV centers studied here, these two environments (oil vs. water) led to clearly different T₁ and T₂. By contrast, when a 20 nm-thick Al₂O₃ layer was deposited to the diamond surface, essentially serving as a blocking material to separate shallow NV centers away from the oil/water environment, similar T₁ and T₂ were obtained as shown in (b). This further corroborated the above-noted conclusion that the Al₂O₃ layer is a cause of the observed finite but significant T₂ decrease after functionalization when imaged in oil. All measurements were performed at 2000 G magnetic field strength. T₂ was measured using Carr-Purcell-Meiboom-Gill (CPMG) pulses. See Table 3 for detailed fitting parameters.

TABLE 3

| | T₂ (μs) | s |
|---|---|---|
| Bare in oil | 14.82 | 0.31 |
| Bare in water | 5.19 | 0.14 |
| Functionalized in oil | 1.91 | 0.47 |
| Functionalized in water | 2.57 | 0.36 |

### Example 6: Functionalization Using TiO₂ Adhesion Layer

Single-crystalline diamonds slabs (2×2×0.5 mm³, Element Six, electronic grade, Catalog No. 145-500-0385) were sonicated in acetone and isopropanol for 5 min each and dried with nitrogen gas before TiO₂ deposition. The deposition of TiO₂ layer was carried out in an Ultratech/Cambridge Savannah ALD System at 100° C. by alternatively delivering and H₂O and Ti(NMe₂)₄ (tetrakis(dimethylamido)titanium) in gas phase, each for 0.015 s followed by 5 s wait time. The growth rate is measured at 0.065 nm/cycle. For 2-nm (10-nm) TiO₂ layer, 30 (150) cycles were used.

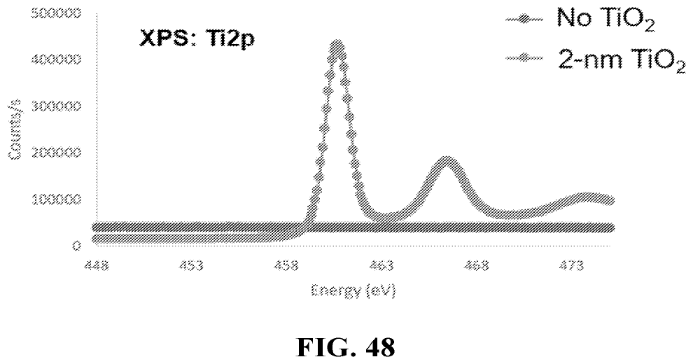

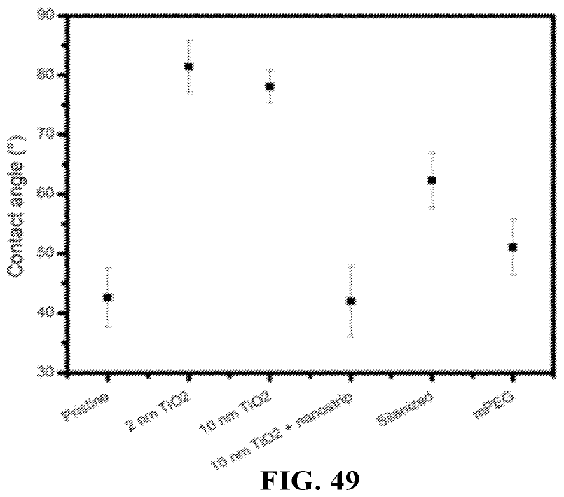

The diamonds were then silanized and PEGylated with mPEG according to Example 2. XPS confirmed the presence of titanium (especially the Ti2p signal) after ALD (FIG. 48). The water contact angle of the surface at each stage of the preparation was also measured. Results are shown in FIG. 49.

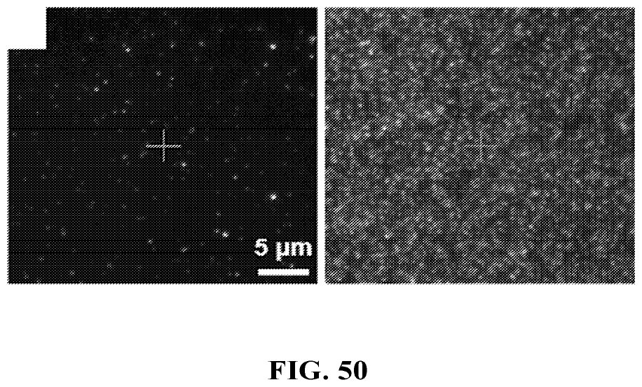

TiO₂-coated glass coverslips were coated with mPEG, or mPEG doped with 0.3% biotinPEG. The PEGylated coverslips were incubated in SA-488 dissolved in phosphate-buffered saline (PBS) for 20 min under room temperature. As shown in FIG. 50, fluorescence microscopy images showed that optically resolvable individual SA-488 molecules were attached on the biotinPEG-doped coating, while the non-specific binding of SA-488 on coverslips coated solely with mPEG was negligible.

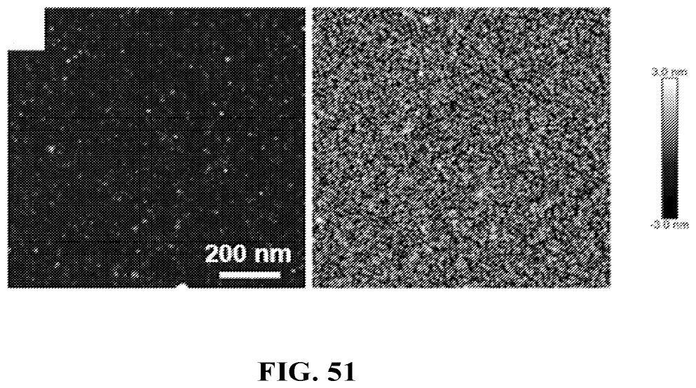

TiO₂-coated silicon wafer ships were coated with mPEG, or mPEG doped with 50% biotinPEG. The PEGylated chips were incubated in SA-488 dissolved in phosphate-buffered saline (PBS) for 20 min under room temperature. As shown in FIG. 51, AFM showed negligible SA-488 protein on the surface of mPEG-coated chips, while SA-488 was readily detected on the biotinPEG-doped coating.

### Items

Certain embodiments of the present disclosure are provided in the following list of items.

Item 1. A device comprising:
a substrate having a top surface, wherein the substrate further comprises one or more color centers in proximity to the top surface;
an optional adhesion layer disposed on the top surface of the substrate, wherein the adhesion layer comprises an oxide;
a functionalized layer configured to contact a sample, wherein the functionalized layer comprises one or more capture agents configured to capture a target; and
an interlayer disposed beneath the functionalized layer.

Item 2. The device of item 1, wherein the substrate comprises a diamond, and wherein the one or more color centers comprise a nitrogen vacancy in the diamond.

Item 3. The device of item 2, wherein the top surface of the substrate comprises an oxygen-terminated surface of the diamond.

Item 4. The device of any of items 1-3, wherein the one or more color centers are disposed at a depth of less than about 100 nm or less than about 10 nm from the top surface.

Item 5. The device of any of items 1-3, wherein the one or more color centers are disposed at a depth of about 2 to about 10 nm from the top surface.

Item 6. The device of any of items 1-5, wherein the adhesion layer is present, and wherein the oxide of the adhesion layer comprises a silanizable oxide.

Item 7. The device of any of items 1-5, wherein the adhesion layer is present, and wherein the oxide of the adhesion layer comprises an aluminum oxide, a silicon oxide, or a titanium oxide.

Item 8. The device of any of items 1-5, wherein the adhesion layer is present, and wherein the oxide of the adhesion layer comprises a titanium oxide.

Item 9. The device of any of items 1-8, wherein the adhesion layer is present, and wherein the oxide comprises a patterned oxide.

Item 10. The device of any of items 1-9, further comprising an active area and an inactive area.

Item 11. The device of item 10, wherein the active area comprises the functionalized layer and the adhesion layer, and the inactive area lacks the functionalized layer and the adhesion layer.

Item 12. The device of item 10, wherein the active area comprises one or more active sites, and wherein the inactive area lacks active sites

Item 13. The device of any of items 1-12, wherein the functionalized layer comprises a poly(ethylene glycol) group, a perfluoroalkylene group, a perfluoroalkyleneoxy group, an alkylene group, a fluoroalkylene group, or a heteroalkylene group.

Item 14. The device of item 13, wherein the poly(ethylene glycol) group comprises —(CH₂CH₂O)ₙ—, in which n is an integer of 3 to 200.

Item 15. The device of item 14, in which n is an integer of 5 to 15.

Item 16. The device of item 14, in which n is an integer of 25 to 150.

Item 17. The device of item 13, wherein the perfluoroalkylene group comprises —C_{f}F_{2f}—, in which f is an integer of 1 to 12.

Item 18. The device of item 13, wherein the perfluoroalkyleneoxy group comprises —OC_{f}F_{2f}— or —C_{f}F_{2f}O—, in which f is an integer of 1 to 12.

Item 19. The device of any of items 13-18, wherein the functionalized layer comprises a monolayer.

Item 20. The device of any of items 1-19, wherein the functionalized layer is configured to provide a biocompatible surface to the sample.

Item 21. The device of any of items 1-20, wherein the one or more capture agents are selected from the group consisting of a nucleic acid, a peptide, a protein, a cofactor, a receptor, an enzyme, an antibody, an affibody, a lectin, and a click chemistry moiety, or a combination thereof.

Item 22. The device of item 21, wherein the peptide comprises a polyhistidine or a polyglycine.

Item 23. The device of item 21, wherein the protein comprises avidin, streptavidin, or neutravidin.

Item 24. The device of item 21, wherein the cofactor comprises biotin.

Item 25. The device of item 21, wherein the cofactor comprises Ni²⁺.

Item 26. The device of item 21, wherein the click chemistry moiety comprises an azido group, an alkynyl group, a cycloalkynyl group, a dienophile group, or a diene group.

Item 27. The device of item 21, wherein the click chemistry moiety comprises a maleimide group or a thiol group.

Item 28. The device of any of items 1-27, wherein the target comprises a biomolecule or a tagged biomolecule, and wherein the biomolecule comprises a nucleic acid, a peptide, a protein, a receptor, a ligand, or a cell.

Item 29. The device of items 1-28, wherein the interlayer comprises an alkylene group or a heteroalkylene group.

Item 30. The device of item 29, wherein the alkylene group comprises a C₁₋₁₂ alkylene.

Item 31. The device of item 29, wherein the heteroalkylene group comprises -Ak-NR^{N1}—, -Ak-O—, [symbol] Si-Ak-NR^{N1}-Ak-NR^{N1}—, [symbol] Si-Ak-, [symbol] Si-Ak-NR^{N1}—, or [symbol] Si-Ak-O—, in which Ak is C₁₋₂ alkylene and R^{N1} is H or C₁₋₆ alkyl.

Item 32. The device of any of items 1-31, wherein a thickness of the adhesion layer, the interlayer and the functionalized layer, taken together, is less than about 10 nm.

Item 33. The device of any of items 1-32, wherein an average number of capture agents present per μm² of the functionalized layer is less than 10.

Item 34. The device of any of items 1-32, wherein an average number of capture agents present per μm² of the functionalized layer is about 0.01 to about 5.

Item 35. The device of any of items 1-34, further comprising:
a source configured to irradiate the substrate and/or the one or more color centers; and
a detector configured to detect one or more output signals emitted from the substrate upon being irradiated.

Item 36. A method of detecting a target, the method comprising:
providing a sample to an active area of the device of any of items 1-35;
irradiating the device to excite the one or more color centers; and
detecting one or more output signals emitted from the substrate upon being irradiated.

Item 37. The method of item 35, wherein the sample comprises a biomolecule, a cell, and/or a physiological buffer.

Item 38. A method of preparing a device, the method comprising:
depositing an adhesion layer on a top surface of a substrate, wherein the substrate comprises one or more color centers in proximity to the top surface;
reacting a top surface of the adhesion layer with a silanizing agent to provide an interlayer, wherein a top surface of the interlayer comprises a reactive moiety; and
attaching a functionalized layer by way of the reactive moiety within the interlayer, wherein the functionalized layer comprises one or more capture agents configured to capture a target.

Item 39. The method of item 38, wherein said depositing comprises atomic layer deposition, chemical vapor deposition, physical vapor deposition, or a plasma-enhanced form thereof.

Item 40. The method of item 38 or item 39, wherein the substrate comprises a diamond, and wherein the top surface of the substrate comprises an oxygen-terminated surface of the diamond.

Item 41. The method of items 38-40, wherein said attaching comprises:
providing a linking group (e.g., a poly(ethylene glycol) group, a perfluoroalkylene group, a perfluoroalkyleneoxy group, an alkylene group, a fluoroalkylene group, or a heteroalkylene group) optionally comprising the one or more capture agents.

Item 42. The method of item 41, wherein said attaching comprises:
providing a mixture of a first linking group (e.g., a first poly(ethylene glycol) group, perfluoroalkylene group, perfluoroalkyleneoxy group, alkylene group, fluoroalkylene group, or heteroalkylene group) comprising the one or more capture agents and a second linking group (e.g., a second poly(ethylene glycol) group, perfluoroalkylene group, perfluoroalkyleneoxy group, alkylene group, fluoroalkylene group, or heteroalkylene group) that lacks the one or more capture agents.

Item 43. The method of any of items 38-40, wherein said attaching comprises:
providing a linking group (e.g., a poly(ethylene glycol) group, perfluoroalkylene group, perfluoroalkyleneoxy group, alkylene group, fluoroalkylene group, or heteroalkylene group) having a further reactive moiety, and
providing one or more capture reagents to react with the further reactive moiety.

Item 44. The method of any of items 38-40, wherein said attaching comprises:
providing a mixture of a first linking group (e.g., a first poly(ethylene glycol) group, perfluoroalkylene group, perfluoroalkyleneoxy group, alkylene group, fluoroalkylene group, or heteroalkylene group) having a further reactive moiety and a second linking group (e.g., a second poly(ethylene glycol) group, perfluoroalkylene group, perfluoroalkyleneoxy group, alkylene group, fluoroalkylene group, or heteroalkylene group) that lacks the further reactive moiety, and
providing one or more capture reagents to react with the further reactive moiety.

Item 45. The device of any of items 1-35, prepared by the method of any of items 38-44.

### OTHER EMBODIMENTS

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are within the claims.

### CONCLUSION

Although the foregoing embodiments have been described in some detail for purposes of clarity of understanding, it will be apparent that certain changes and modifications may be practiced within the scope of the appended claims. It should be noted that there are many alternative ways of implementing the processes, systems, and apparatus of the present embodiments. Accordingly, the present embodiments are to be considered as illustrative and not restrictive, and the embodiments are not to be limited to the details given herein.

1. A device comprising:
a substrate having a top surface, wherein the substrate further comprises one or more color centers in proximity to the top surface;
an optional adhesion layer disposed on the top surface of the substrate, wherein the adhesion layer comprises an oxide;
a functionalized layer configured to contact a sample, wherein the functionalized layer comprises one or more capture agents configured to capture a target; and
an interlayer disposed beneath the functionalized layer.

2. The device of claim 1, wherein the substrate comprises a diamond, and wherein the one or more color centers comprise a nitrogen vacancy in the diamond, and optionally wherein the top surface of the substrate comprises an oxygen-terminated surface of the diamond.

3. (canceled)

4. The device of claim 1, wherein the one or more color centers are disposed at a depth of less than about 100 nm from the top surface.

5. The device of claim 1, wherein the adhesion layer is present, and wherein the oxide of the adhesion layer comprises a silanizable oxide, an aluminum oxide, a silicon oxide, a titanium oxide, or a patterned oxide.

6-9. (canceled)

10. The device of claim 1, further comprising an active area and an inactive area, wherein the active area comprises the functionalized layer and the adhesion layer, and wherein the inactive area lacks the functionalized layer and the adhesion layer.

11. (canceled)

12. The device of claim 1, wherein the functionalized layer comprises a poly(ethylene glycol) group, a perfluoroalkylene group, a perfluoroalkyleneoxy group, an alkylene group, a fluoroalkylene group, or a heteroalkylene group.

13-17. (canceled)

18. The device of claim 12, wherein the functionalized layer comprises a monolayer, or wherein the functionalized layer is configured to provide a biocompatible surface to the sample.

19. (canceled)

20. The device of claim 1, wherein the one or more capture agents are selected from the group consisting of a nucleic acid, a peptide, a protein, a cofactor, a receptor, an enzyme, an antibody, an affibody, a lectin, and a click chemistry moiety, or a combination thereof.

21-26. (canceled)

27. The device of claim 1, wherein the target comprises a biomolecule or a tagged biomolecule, and wherein the biomolecule comprises a nucleic acid, a peptide, a protein, a receptor, a ligand, or a cell.

28. The device of claim 1, wherein the interlayer comprises an alkylene group or a heteroalkylene group.

29-30. (canceled)

31. The device of claim 1, wherein a thickness of the adhesion layer if present, the interlayer, and the functionalized layer, taken together, is less than about 10 nm.

32. The device of claim 1, wherein an average number of capture agents present per μm² of the functionalized layer is less than 10 or is about 0.01 to about 5.

33. (canceled)

34. The device of claim 1, further comprising:
a source configured to irradiate the substrate and/or the one or more color centers; and
a detector configured to detect one or more output signals emitted from the substrate upon being irradiated.

35. A method of detecting a target, the method comprising:
providing a sample to an active area of the device of claim 1;
irradiating the device to excite the one or more color centers; and
detecting one or more output signals emitted from the substrate upon being irradiated.

36. (canceled)

37. A method of preparing a device, the method comprising:
depositing an adhesion layer on a top surface of a substrate, wherein the substrate comprises one or more color centers in proximity to the top surface;
reacting a top surface of the adhesion layer with a silanizing agent to provide an interlayer, wherein a top surface of the interlayer comprises a reactive moiety; and
attaching a functionalized layer by way of the reactive moiety within the interlayer, wherein the functionalized layer comprises one or more capture agents configured to capture a target.

38. (canceled)

39. The method of claim 37, wherein the substrate comprises a diamond, and wherein the top surface of the substrate comprises an oxygen-terminated surface of the diamond.

40. The method of claim 37, wherein said attaching comprises:
(i) providing a poly(ethylene glycol) group optionally comprising the one or more capture agents; or
(ii) providing a poly(ethylene glycol) group having a further reactive moiety and providing one or more capture reagents to react with the further reactive moiety.

41. The method of claim 40, wherein said attaching comprises:
(i) providing a mixture of a first poly(ethylene glycol) group comprising the one or more capture agents and a second poly(ethylene glycol) group that lacks the one or more capture agents; or
(ii) providing a mixture of a first poly(ethylene glycol) group having a further reactive moiety and a second poly(ethylene glycol) group that lacks the further reactive moiety; and providing one or more capture reagents to react with the further reactive moiety.

42-43. (canceled)

44. A device prepared by the method of claim 37.

45. The device of claim 1, wherein the device is configured employ one or more dynamical decoupling sequences to decouple the device from low frequency noise.


SEQUENCE LISTING (4 sequences)

SEQ ID NO: 1 - DNA, 40 bp, synthetic construct
  source 1..40: mol_type = other DNA
ttttttttttagtccgtggtagggcaggttggggtgactt

SEQ ID NO: 2 - protein, 8 aa, synthetic construct
DYKDDDDK

SEQ ID NO: 3 - protein, 8 aa, synthetic construct
WSHPQFEK

SEQ ID NO: 4 - protein, 5 aa, synthetic construct
LPXTG